(19) United States
(12) Patent Application Publication
(10) Pub. No.: US 2012/0238448 A1
(43) Pub. Date: Sep. 20, 2012

(54) METHOD FOR TREATING PHYTOPATHOGENIC MICROORGANISMS USING SURFACE-MODIFIED NANOPARTICULATE COPPER SALTS

(75) Inventors: Karl-Heinrich Schneider, Kleinkarlbach (DE); Andrey Karpov, Mannheim (DE); Hartwig Voss, Frankenthal (DE); Sarah Dunker, Waldsee (DE); Michael Merk, Arese (IT); Alexander Kopf, Speyer (DE); Shoichi Kondo, Kawaguchi-shi (JP)

(73) Assignee: BASF SE, Ludwigshafen (DE)

(21) Appl. No.: 13/512,926

(22) PCT Filed: Nov. 29, 2010
(86) PCT No.: PCT/EP2010/068354

§ 371 Date: May 31, 2012

(30) Foreign Application Priority Data
Dec. 2, 2009 (EP) 09177760.7

(51) Int. Cl. A01N 59/20; A01N 55/02; A01P 7/04; A01P 7/02; A01P 21/00; A01P 13/00; A01N 25/34; A01P 1/00; B82Y 5/00

(52) U.S. Cl. 504/121; 424/400; 514/499; 424/638; 424/633; 424/632; 504/187; 504/190; 977/773

(57) ABSTRACT

The present invention relates to a method for controlling phytopathogenic microorganisms by treating the crop to be protected, the soil or the plant propagation material with an effective amount of copper salt particles which comprise a water-soluble polymer and which have a primary particle diameter of from 1 to 200 nm. The invention also relates to an aqueous suspension of the abovementioned copper salt particles and the use of this suspension in crop protection.

The present invention relates to a method for controlling phytopathogenic micro-organisms by treating the crop to be protected, the soil or the plant reproduction material with an effective amount of copper salt particles which comprise a water-soluble polymer and which have a primary particle diameter of from 1 to 200 nm. The invention also relates to an aqueous suspension of the abovementioned copper salt particles and the use of this suspension in crop protection. Combinations of preferred features with other preferred features are encompassed by the present invention.

Crop protection agents based on copper compounds have long been known and valuable aids for controlling fungi in agriculture. One of the oldest examples is Bordeaux mixture, a suspension of quicklime (CaO) in an aqueous copper sulfate solution. Even in ecological cultivation, these chemicals are recognized as fungicides and are permitted. The problem since time immemorial is the required, high application rate (generally from 500 to 1500 g of copper per hectare), which may constitute contamination of the environment (e.g. by copper accumulation in the soil) and of the useful plants to be protected.

US 2002/0112407 discloses the production of inorganic nanoparticulate particles having a mean size of from 2 to 500 nm, preferably<100 nm (determined by dynamic light scattering, DLS), by partial or complete alkaline hydrolysis of at least one metal compound which is either dissolved in an aqueous medium or suspended in nanoparticulate form, in the presence of water-soluble comb polymers. The use of the particles thus obtained in fungicidal or biocidal dispersions is likewise disclosed. A disadvantage of this method is that at least partly intensely colored metal oxides, hydroxides or oxide/hydroxides are always obtained and hence metal compounds free of metal hydroxide/oxide are not obtainable.

WO 2010/003870 discloses a method for the preparation of surface-modified nanoparticulate copper compounds. There, an aqueous solution of copper ions and a solution of anions forming a precipitate with copper are mixed in the presence of a polymer and copper salts are thus precipitated. The use of nanoparticles and of the aqueous dispersion comprising the nanoparticles as an antimicrobial active substance is likewise disclosed.

WO 2005/110692 describes aqueous suspensions comprising microparticulate copper compounds (e.g. copper hydroxide, copper carbonate) for wood preservation. The suspensions having mean particle sizes in the range of from about 200 nm to about 400 nm were prepared by wet milling in the presence of dispersants.

The wood preservative preparations disclosed in WO 2006/042128 comprise, inter alia, sparingly soluble copper compounds which are likewise brought into finely divided form by milling.

US 2005/0256026 discloses an aqueous slurry of copper salts, quaternary ammonium salts and dispersant.

A disadvantage of milling methods is that particles having a mean particle size of <100 nm were obtainable only with great effort and by means of a very large energy input.

It was therefore an object of the present invention to provide a method for controlling phytopathogenic microorganisms, in particular fungi, in which the plants to be protected from fungal attack, the soil or the seeds can be treated with a copper-containing formulation at as low an application rate as possible. The copper-containing formulation for use in the method should be capable of being prepared as easily and economically as possible. In this method, the plants or seeds to be protected should be brought into contact with as small an amount of copper compounds as possible and/or should be damaged as little as possible. The method and the formulation should be suitable in particular for use in cultivation of grapevines, fruit and vegetables.

The object was achieved by a method for controlling phytopathogenic fungi by treating the crop to be protected, the soil or the plant propagation material with an effective amount of copper salt particles which comprise a water-soluble polymer and have a particle diameter of from 1 to 200 nm, the copper salt comprising an anion which is not hydroxide and forms a precipitate with copper ions. Preferably, the crop to be protected or the plant propagation material is treated, in particular the crop to be protected.

The copper salt comprises an anion which is not hydroxide and forms a precipitate with copper ions (in particular in water at 20° C. and a concentration of 0.1 mol/l of the copper salt, no later than 1 h after mixing of the ions). Preferred anions are anions of phosphoric acid, carbonic acid, boric acid, sulfurous acid or anions of organic acids, such as oxalic acid, benzoic acid, maleic acid, etc., and polyborates, such as B₄O₇²⁻. The anions carbonate, phosphate, hydrogen phosphate, oxalate, borate and tetraborate ions are particularly preferred, in particular oxalate and carbonate anions.

In addition to the anion which is not hydroxide and forms a precipitate with copper ions, the copper salt may comprise further anions. Suitable further anions are also the abovementioned anions which are not hydroxide and form a precipitate with copper ions. Preferred further anions are anions of mineral acids, such as hydrochloric acid, sulfuric acid, phosphoric acid, carbonic acid, boric acid, sulfurous acid, etc., or anions of organic acids, such as oxalic acid, benzoic acid, maleic acid, etc., and polyborates, such as B4_{O}₇²⁻ or hydroxide (OH⁻). The further anions are preferably hydroxide anions.

In a further preferred embodiment, the further anions are hydroxide anions or the polycarboxylates described below (preferably polycarboxylates based on acrylic acid, methacrylic acid, maleic acid or mixtures thereof). In the case of polycarboxylates, some of the carboxylate groups may be present in the polymer in anionic form and, as anions, therefore form salts.

Preferably, the copper salt comprises carbonate anions and hydroxide anions.

In addition to copper ions, the copper salt may also comprise further metal ions, for example ions of alkaline earth metals or transition metals, preferably magnesium, calcium, chromium, cobalt, nickel, zinc or silver ions, particularly preferably zinc or silver ions. The further metal ions are present in smaller numbers than the copper ions. Preferably, no further metal ions are present.

The copper salt may also comprise water of crystallization.

In general, a distinction is made in the case of particles between the primary and the secondary particle diameter. A plurality of smaller particles (having a primary particle diameter) may agglomerate to form a larger particle (having a secondary particle diameter). The secondary particle diameter can therefore often also be designated as aggregate size or agglomerate size. The secondary particle diameter can be determined, for example, by dynamic light scattering, but the primary particle diameter cannot be determined. During the purification of particles, a change in the secondary particle diameter may occur if, for example, the primary particles combine to form increasingly larger aggregates of primary particles.

The primary particle diameter of the copper salt particles is in general in the range from 0.1 to 200 nm, preferably from 1 to 100 nm, in particular from 1 to 50 nm. The primary particle diameter is preferably determined via transmission electron microscopy (TEM).

The secondary particle diameter usually designates the mean particle diameter which is determined according to the volume fraction from the particle size distributions. The particle size distributions can be measured by light scattering (for example on a Zetasizer Nano S apparatus from Malvern Instruments). The secondary particle diameter of the copper salt particles is in general in the range from 0.1 to 300 nm, preferably from 1 to 200 nm.

The copper salt particles are preferably amorphous. Amorphous means that the molecular building blocks of a homogeneous solid are not arranged in crystal latices. An amorphous form of the copper salt particles means that it is substantially free of crystalline copper salt, preferably from 80 to 100% by weight, in particular from 90 to 100% by weight, of the copper salt being present in amorphous form. Amorphous forms can be distinguished from crystalline forms by various methods, for example by microscopic examination in polarized light, differential scanning calorimetry, X-ray diffraction or solubility comparisons. X-ray diffraction is preferred. The choice of the method depends, for example, on the fineness of the particles.

The water-soluble polymer may be present in the copper salt particles in various ways. In one embodiment, it is possible for the surface of the particles to be modified with the polymer. There, the polymer is present at least partly on the surface of the particles. In a further embodiment, the polymer is present partly internally in the copper salt particles. Particularly with the use of anionic, water-soluble polymers (such as the polycarboxalates), the polymers can partly form salts with the copper ions. Usually, the water-soluble polymer does not form a chemically crosslinked capsule shell around the copper salt.

The water-soluble polymers may be anionic, cationic, nonionic or zwitterionic polymers. The molecular weight is in general in the range of from about 800 to about 500 000 g/mol, preferably in the range of from about 1000 to about 30 000 g/mol. In a further embodiment, the molecular weight is in the range of from 5000 to about 50 000 g/mol and preferably in the range of from 10 000 to 40 000 g/mol. There may be homo- or copolymers and their molecular structure may be either linear or branched. Water-soluble polymers having a comb structure are preferred.

Suitable monomers from which the water-soluble polymers to be used according to the invention are obtainable comprise, for example, α, β-unsaturated carboxylic acids and esters, amides and nitriles thereof, N-vinylcarboxamides, alkylene oxides, unsaturated sulfonic acids and phosphonic acids and amino acids.

In an embodiment of the invention, polycarboxylates are used as water-soluble polymers. In this invention, polycarboxylates are polymers based on at least one α, β-unsaturated carboxylic acid, for example acrylic acid, methacrylic acid, dimethacrylic acid, ethacrylic acid, maleic acid, citraconic acid, methylenemalonic acid, crotonic acid, isocrotonic acid, fumaric acid, mesaconic acid and itaconic acid. Polycarboxylates based on acrylic acid, methacrylic acid, maleic acid or mixtures thereof are preferably used.

The proportion of the at least one α, β-unsaturated carboxylic acid in the polycarboxylates is as a rule in the range from 20 to 100 mol %, preferably in the range from 50 to 100 mol %, particularly preferably in the range from 75 to 100 mol %.

The polycarboxylates to be used according to the invention can be used both in the form of the free acid and partly or completely neutralized in the form of their alkali metal, alkaline earth metal or ammonium salts. However, they can also be used as salts of the respective polycarboxylic acid and triethylamine, ethanolamine, diethanolamine, triethanolamine, morpholine, diethylenetriamine or tetraethylene-pentamine.

In addition to the at least one α, β-unsaturated carboxylic acid, the polycarboxylates may comprise further comonomers which are incorporated in the form of polymerized units in the polymer chain, for example the esters, amides and nitriles of the abovementioned carboxylic acids, such as methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, methyl maleate, dimethyl maleate, monoethyl maleate, diethyl maleate, 2-ethylhexyl acrylate, 2-ethylhexyl methacrylate, acrylamide, methacrylamide, N-dimethylacrylamide, N-tert-butylacryl-amide, acrylonitrile, methacrylonitrile, dimethylaminoethyl acrylate, diethylaminoethyl acrylate, diethylaminoethyl methacrylate and the salts of the last-mentioned basic monomers with carboxylic acids or mineral acids and the quaternized products of the basic (meth)acrylates.

Allylacetic acid, vinylacetic acid, acrylamidoglycolic acid, vinylsulfonic acid, allylsulfonic acid, methallylsulfonic acid, styrenesulfonic acid, 3-sulfopropyl acrylate, 3-sulfopropyl methacrylate or acrylamidomethylpropanesulfonic acid and monomers comprising phosphonic acid groups, such as vinylphosphonic acid, allylphosphonic acid or acrylamidomethylpropanephosphonic acid, are also suitable as further comonomers which can be incorporated in the form of polymerized units. The monomers comprising acid groups can be used in the polymerization in the form of the free acid groups and in a form partly or completely neutralized with bases.

Further suitable copolymerizable compounds are N-vinylcaprolactam, N-vinylimidazole, N-vinyl-2-methylimidazole, N-vinyl-4-methylimidazole, vinyl acetate, vinyl propionate, isobutene, styrene, ethylene oxide, propylene oxide or ethyleneimine and compounds having more than one polymerizable double bond, such as, for example, diallyl-ammonium chloride, ethylene glycol dimethacrylate, diethylene glycol diacrylate, allyl methacrylate, trimethylolpropane triacrylate, triallylamine, tetraallyloxyethane, triallyl cyanurate, diallyl maleate, tetraallylethylenediamine, divinylideneurea, pentaerythrityl diallyl ether, pentaerythrityl triallyl ether and pentaerythrityl tetraallyl ether, N,N″-methylenebisacrylamide or N,N″-methylenebismethacrylamide.

It is of course also possible to use mixtures of said comonomers. For example, mixtures of from 50 to 100 mol % of acrylic acid and from 0 to 50 mol % of one or more of said comonomers are suitable for the preparation of the polycarboxylates according to the invention.

Numerous polycarboxylates to be used according to the invention are commercially available under the trade name Sokalan® (from BASF SE).

In further embodiments of the invention, the water-soluble polymer is polyaspartic acid, polyvinylpyrrolidone or a copolymer of N-vinylamide, for example N-vinylpyrrolidone, and at least one further monomer comprising a polymerizable group, for example with monoethylenically unsaturated C₃-C₈-carboxylic acids, such as acrylic acid, methacrylic acid, C₈-C₃₀-alkyl esters of monoethylenically unsaturated C₃-C₈-carboxylic acids, vinyl esters of aliphatic C₈-C₃₀-carboxylic acids and/or N-alkyl- or N,N-dialkyl-substituted amides of acrylic acid or of methacrylic acid having C₈-C₁₈-alkyl radicals.

In a preferred embodiment of the process according to the invention, the water-soluble polymer used is polyaspartic acid. In the context of the present invention, the term polyaspartic acid comprises both the free acid and the salts of polyaspartic acid, e.g. sodium, potassium, lithium, magnesium, calcium, ammonium, alkylammonium, zinc and iron salts or mixtures thereof.

In a further embodiment of the invention, nonionic water-soluble polymers are used. In the context of this invention, a nonionic water-soluble polymer is a surface-active substance whose chemical structure comprises from 2 to 1000 —CH₂CH₂O— groups, preferably from 2 to 200 —CH₂CH₂O— groups, particularly preferably from 2 to 80 —CH₂CH₂O— groups. These groups form, for example, by an addition reaction of a corresponding number of ethylene oxide molecules with substrates containing hydroxyl or carboxyl groups and as a rule form one or more cohesive ethylene glycol chains whose chemical structure corresponds to the formula —(CH₂CH₂O—)ₙ— where n is from about 2 to about 80.

In a preferred embodiment of the invention, the nonionic water-soluble polymer used is at least one substance from one of the following groups:

Adducts of from 2 to 80 mol of ethylene oxide and optionally from 1 to 15 mol of propylene oxide with
alkylphenols having 1 to 5 carbon atoms in the alkyl group,
glycerol mono- and diesters, sorbitol mono- and diesters and sorbitan mono- and diesters of saturated and unsaturated fatty acids having 6 to 22 carbon atoms,
alkylmono- and -oligoglycosides having 1 to 5 carbon atoms in the alkyl radical,
acetic acid,
lactic acid,
glycerol,
polyglycerol,
pentaerythritol,
dipentaerythritol,
sucrose,
sugar alcohols (e.g. sorbitol),
alkylglucosides (e.g. methylglucoside, butylglucoside, laurylglucoside),
polyglucosides (e.g. cellulose),

Polyalkylene glycols whose structure comprises from 2 to 80 ethylene glycol units.

In a particularly preferred embodiment of the invention, the nonionic water-soluble polymer used is at least one substance from one of the following groups:

Adducts of from 2 to 80 mol of ethylene oxide with
alkylphenols having 1 to 5 carbon atoms in the alkyl group,
glycerol, and
alkylglucosides.

Numerous nonionic water-soluble polymers to be used according to the invention are commercially available under the trade name Cremophor® (from BASF SE).

The ethylene oxide adducts in technical quality may still comprise a small proportion of the substrates listed above by way of example and containing free hydroxyl or carboxyl groups. As a rule, this proportion is less than 20% by weight, preferably less than 5% by weight, based on the total amount of the nonionic water-soluble polymer.

In a further embodiment of the invention, water-soluble polymers used are homo- and copolymers of N-vinylcarboxamides. These polymers are prepared by homo- or copolymerization of, for example, N-vinylformamide, N-vinylacetamide, N-alkyl-N-vinylformamide or N-alkyl-N-vinylacetamide. Among the N-vinylcarboxamides, N-vinylformamide is preferably used, homopolymers of N-vinylformamide being particularly preferred.

The water-soluble N-vinylcarboxamide polymers to be used according to the invention may optionally also comprise from 0 to 80, preferably from 5 to 30% by weight of comonomers incorporated in the form of polymerized units, in each case based on the total composition of the polymers, in addition to from 100 to 20% by weight of the N-vinylcarboxamides. The comonomers are, for example, monoethylenically unsaturated carboxylic acids having 3 to 8 carbon atoms, such as acrylic acid, methacrylic acid, dimethacrylic acid, ethacrylic acid, maleic acid, citraconic acid, methylenemalonic acid, allylacetic acid, vinylacetic acid, crotonic acid, fumaric acid, mesaconic acid and itaconic acid. From this group of monomers, acrylic acid, methacrylic acid, maleic acid or mixtures of said carboxylic acids are preferably used. The monoethylenically unsaturated carboxylic acids are used in the copolymerization either in the form of the free acids or in the form of their alkali metal, alkaline earth metal or ammonium salts. However, they can also be used as salts of the respective acid and triethylamine, ethanolamine, diethanolamine, triethanolamine, morpholine, diethylenetriamine or tetraethylenepentamine.

Further suitable comonomers are, for example, the esters, amides and nitriles of the abovementioned carboxylic acids, e.g. methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, acrylamide, methacrylamide, N-dimethylacrylamide, N-tert-butyl-acrylamide, acrylonitrile, methacrylonitrile, dimethylaminoethyl acrylate, diethylamino-ethyl acrylate, diethylaminoethyl methacrylate and the salts of the last-mentioned basic monomers with carboxylic acids or mineral acids and the quaternized products of the basic (meth)acrylates. Acrylamide or methacrylamide is preferably used.

Acrylamidoglycolic acid, vinylsulfonic acid, allylsulfonic acid, methallylsulfonic acid, styrenesulfonic acid, 3-sulfopropyl acrylate, 3-sulfopropyl methacrylate or acrylamido-methylpropanesulfonic acid and monomers comprising phosphonic acid groups, such as vinylphosphonic acid, allylphosphonic acid or acrylamidomethanepropane-phosphonic acid, are also suitable as further comonomers which can be incorporated in the form of polymerized units. The monomers comprising acid groups can be used in the polymerization in the form of the free acid groups and in a form partly or completely neutralized with bases.

Further suitable copolymerizable compounds are N-vinylpyrrolidone, N-vinylcapro-lactam, N-vinylimidazole, N-vinyl-2-methylimidazole, N-vinyl-4-methylimidazole, vinyl acetate, vinyl propionate, isobutene, styrene, ethylene oxide, propylene oxide or ethyleneimine and compounds having more than one polymerizable double bond such as, for example, diallylammonium chloride, ethylene glycol dimethacrylate, diethylene glycol diacrylate, allyl methacrylate, trimethylolpropane triacrylate, triallylamine, tetraallyloxyethane, triallyl cyanurate, diallyl maleate, tetraallylethylenediamine, divinylideneurea, pentaerythrityl diallyl ether, pentaerythrityl triallyl ether and pentaerythrityl tetraallyl ether, N,N′-methylenebisacrylamide or N,N′-methylenebis-methacrylamide.

It is of course also possible to use mixtures of said comonomers. For example, mixtures of from 50 to 100% by weight of N-vinylformamide and from 0 to 50% by weight of one or more of said comonomers are suitable for the preparation of the water-soluble polymers according to the invention.

If said comonomers do not give water-soluble polymers when polymerized alone, the polymers comprising N-vinylcarboxamide units comprise these comonomers incorporated in the form of polymerized units only in amounts such that the copolymers are still water-soluble.

In a preferred embodiment of the invention, nonionic water-soluble polymers having a comb-like molecular structure are used, which polymers are obtained, for example, by copolymerization of monomer mixtures comprising macromonomers. The structure of the nonionic water-soluble polymers having a comb-like molecular structure can be described, for example, as a complex-forming polymer backbone having anionic and/or cationic groups and hydrophilic side chains or as a neutral hydrophilic polymer backbone having complex-forming anionic and/or cationic groups.

In the context of this invention, macromonomers are understood as meaning substances which, at a molecular weight of preferably less than 500 000 D, in particular in the range from 300 to 100 000 D, particularly preferably in the range from 500 to 20 000 D, very particularly preferably in the range from 800 to 15 000 D, have a substantially linear molecular structure and carry a polymerizable terminal group at one end.

In a preferred embodiment of the invention, macromonomers which are based on polyalkylene glycols and are provided with a polymerizable terminal group at one end are used for the preparation of the water-soluble polymers having a comb-like molecular structure. Said polymerizable terminal group may be, for example, a vinyl, allyl, (meth)acrylic acid or (meth)acrylamide group, the corresponding macromonomers being described by the following formulae (formula (VI) being preferred):

CH_{2═CR}²—P, (II)

CH_{2═CH—CH}₂—P, (III)

CH_{2═CH—CH}₂—NH—R³—P, (IV)

CH_{2═CH—CH}₂—CO—P, (V)

CH_{2═CR}²—CO—P, (VI)

CH_{2═CR}²—CO—NH—R³—P, (VII)

CH_{2═CR}²—CO—O—R³—P, (VIII)

where

R²=is H or methyl,

R³ is as defined below and

P is a polyalkylene glycol radical of the general formula

P=—{—O—(R³O)ᵤ—R⁴O)ᵥ—(R⁵O)_{w}—[-A-(R⁶O)ₓ—(R⁷O)_{y}—(R⁸O)_{z}-]ₛ—R⁹}ₙ

in which the variables, independently of one another, have the following meaning:

R⁹ is hydrogen, NH₂, C₁-C₈-alkyl, R¹⁰—C(═O)—, R¹⁰—NH—C(═O)—;

R³ to R⁸ are —(CH₂)₂—, —(CH₂)₃—, —(CH₂)₄—, —CH₂—CH(CH₃)—, —CH₂—CH(CH₂—CH₃)—, —CH₂—CHOR¹¹—CH₂—;

R¹⁰ is C₁-C₈-alkyl;

R¹¹ is hydrogen, C₁-C₈-alkyl, R¹⁰—C(═O)—;

A —C(═O)—O—, —C(═O)—B—C(═O)—O—, —C(═O)—NH—B—NH—C(═O)—O—;

B is —(CH₂)ₜ—, arylene, optionally substituted;

n is from 1 to 8;

s is from 0 to 500, preferably from 0 to 20;

t is from 1 to 8;

u is from 1 to 5000, preferably from 1 to 1000, particularly preferably from 1 to 100;

v is from 0 to 5000, preferably from 0 to 1000;

w is from 0 to 5000, preferably from 0 to 1000;

x is from 1 to 5000, preferably from 1 to 1000;

y is from 0 to 5000, preferably from 0 to 1000; and

z is from 0 to 5000, preferably from 0 to 1000.

Preferred compounds are those in which the polyalkylene glycol radical P is derived from a polyalkylene glycol which has been prepared using ethylene oxide, propylene oxide and butylene oxide and polytetrahydrofuran. Depending on the type of monomer building blocks used here, the result is a polyalkylene glycol radical P having the following structural units: —(CH₂)₂—O—, —(CH₂)₃—O—, —(CH₂)₄—O—, —CH₂—CH(CH₃)—O—, —CH₂—CH(CH₂—CH₃)—O—, —CH₂—CHOR¹¹—CH₂—O—.

The terminal primary hydroxyl group of the polyalkylene glycol radical P (R⁹=H) can be either present in free form or etherified or esterified with alcohols having a chain length of C₁-C₈ or with carboxylic acids having a chain length of C₁-C₈, respectively. However, they can also be exchanged for primary amino groups by reductive amination with hydrogen/ammonia mixtures under pressure or converted into terminal aminopropyl groups by cyanoethylation with acrylonitrile and hydrogenation.

Branched or straight C₁-C₈-alkyl chains, preferably methyl, ethyl, n-propyl, 1-methyl-ethyl, n-butyl, 1-methylpropyl, 2-methylpropyl, 1,1-dimethylethyl, n-pentyl, 1-methyl-butyl, 2-methylbutyl, 3-methylbutyl, 2,2-dimethylpropyl, 1-ethylpropyl, n-hexyl, 1,1-dimethylpropyl, 1,2-dimethylpropyl, 1-methyl pentyl, 2-methylpentyl, 3-methyl pentyl, 4-methylpentyl, 1,1-dimethylbutyl, 1,2-dimethylbutyl, 1,3-dimethylbutyl, 2,2-dimethyl-butyl, 2,3-dimethylbutyl, 3,3-dimethylbutyl, 1-ethylbutyl, 2-ethylbutyl, 1,1,2-trimethyl-propyl, 1,2,2-trimethylpropyl, 1-ethyl-1-methylpropyl, 1-ethyl-2-methylpropyl, n-heptyl, 2-ethylhexyl and n-octyl, may be mentioned as alkyl radicals R⁹ to R¹¹.

Branched or straight C₁-C₆-alkyl chains, particularly preferably C₁-C₄-alkyl chains, may be mentioned as preferred members of the abovementioned alkyl radicals.

These water-soluble polymers having a comb-like molecular structure also comprise as a rule from about 10 to 90, preferably from 25 to 70, % by weight of comonomers which are incorporated in the form of polymerized units and carry deprotonatable groups, in addition to from about 90 to 10% by weight of the macromonomers described. Comonomers may be, for example, monoethylenically unsaturated carboxylic acids having 3 to 8 carbon atoms, such as acrylic acid, methacrylic acid, dimethacrylic acid, ethacrylic acid, maleic acid, citraconic acid, methylenemalonic acid, allylacetic acid, vinylacetic acid, crotonic acid, fumaric acid, mesaconic acid and itaconic acid. From this group of comonomers, acrylic acid, methacrylic acid, maleic acid or mixtures of said carboxylic acids are preferably used. The monoethylenically unsaturated carboxylic acids are used in the copolymerization either in the form of the free acids or in the form of their alkali metal, alkaline earth metal or ammonium salts. However, they may also be used as salts of the respective acid and triethylamine, ethanolamine, diethanolamine, triethanolamine, morpholine, diethylenetriamine or tetraethylenepentamine.

Further suitable comonomers are, for example, the esters, amides and nitriles of the abovementioned carboxylic acids, e.g. methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, acrylamide, methacrylamide, N-di methylacrylamide, N-tert-butyl-acrylamide, acrylonitrile or methacrylonitrile, which, after their incorporation in the form of polymerized units into the water-soluble polymers having a comb-like molecular structure, can be hydrolyzed to give the corresponding free carboxylic acids.

It is of course also possible to use mixtures of said comonomers. The monomers may be present in the copolymers in random distribution or as so-called block polymers.

If said comonomers do not give water-soluble polymers when polymerized alone, the water-soluble polymers comprising macromonomers and having a comb-like molecular structure comprise these comonomers incorporated in the form of polymerized units only in amounts such that they are still water-soluble.

The copper salt particles are preferably obtainable, and are obtainable in particular, by a method comprising the steps:

a) preparation of an aqueous solution comprising copper ions (solution 1) and of an aqueous solution comprising at least one anion which is not hydroxide and forms a precipitate with copper ions (solution 2), at least one of the two solutions 1 and 2 comprising at least one water-soluble polymer,

b) mixing of the solutions 1 and 2 prepared in step a) at a temperature in the range of from 0 to 100° C., the copper salt particles forming with formation of an aqueous dispersion, and

c) optionally concentration of the aqueous dispersion formed and/or removal of byproducts.

Optionally the preparation method may comprise in step d):

d) drying of the surface-modified nanoparticulate copper compounds obtained in step c).

The preparation of the solution 1 described in step a) can be effected, for example, by dissolving the water-soluble copper salt in water or an aqueous solvent mixture. An aqueous solvent mixture may also comprise, for example, water-miscible alcohols, ketones or esters, such as methanol, ethanol, acetone or ethyl acetate, in addition to water. The water content of such a solvent mixture is usually at least 50% by weight, preferably at least 80% by weight. The water-soluble copper salts may be, for example, copper(II) halides, acetates, sulfates or nitrates. Preferred copper salts are copper chloride, copper acetate, copper sulfate and copper nitrate. These salts dissolve in water with formation of copper ions which have a double positive charge and with which six water molecules are associated [Cu(H₂O)₆²⁺]. The concentration of the copper ions in the solution 1 is as a rule in the range of from 0.05 to 2 mol/l, preferably in the range of from 0.1 to 1 mol/l. In addition to the copper ions, solution 1 may also comprise further metal ions (M^{k+}), from which, together with the copper ions, the copper salt particles optionally form in step b). These may be ions of alkaline earth metals or transition metals, preferably magnesium, calcium, chromium, cobalt, nickel, zinc or silver ions, particularly preferably zinc or silver ions. The further metal ions are present in smaller numbers than the copper ions.

Solution 2 may comprise at least one anion which is not hydroxide and forms a precipitate with copper ions. Said anions may be, for example, anions of mineral acids, such as hydrochloric acid, sulfuric acid, phosphoric acid, carbonic acid, boric acid, sulfurous acid, etc., or anions of organic acids, such as oxalic acid, benzoic acid, maleic acid, etc., and polyborates, such as B₄O₇²⁻. In addition, solution 2 can of course additionally comprise hydroxide ions.

In a further embodiment, the anion which forms the precipitate with copper ions may be formed from a precursor compound only in the course of the reaction taking place in step b). The anion is present here in the precursor compound in masked form and is liberated therefrom on mixing of the solutions 1 and 2 and/or by a change in temperature. The precursor compound may be present either in solution 1 or in solution 2 or in both solutions. Dimethyl carbonate may be mentioned as such a precursor compound, from which carbonate ions can be liberated in an alkaline medium. Oxalic acid may be mentioned as a further example of such a precursor compound, from which oxalate anions can be liberated in an alkaline medium. Preferably, solution 1 comprises the precursor compound and solution 2 comprises a reagent (preferably an inorganic base, such as an alkali or alkaline earth metal hydroxide) to liberate the anion which combines with copper ions to form a precipitate. When solution 1 comprises the precursor compound, solution 2 may be free of the anion which combines with copper ions to form a precipitate. A suitable, alternative embodiment of process step a) is as follows: a) preparing an aqueous solution comprising copper ions and a precursor compound of an anion which is not a hydroxide and combines with copper ions to form a precipitate (solution 1), and an aqueous solution comprising a reagent for liberating the anion which combines with copper ions to form a precipitate (solution 2), wherein at least one of the two solutions 1 and 2 comprises at least one water-soluble polymer.

The concentration of the water-soluble polymers in the solutions 1 and/or 2 prepared in step a) of the method is as a rule in the range of from 0.1 to 30 g/l, preferably from 1 to 25 g/l, particularly preferably from 5 to 20 g/l.

In a further preferred form, in general at least 10 g of water-soluble polymer per mole of copper ions, preferably at least 50 g/mol, in particular at least 80 g/mol, are used in step a) of the method. In general, up to 5000 g of water-soluble polymer per mol of copper ions are used, preferably up to 1000 g/mol, in particular at least 700 g/mol.

The mixing of the two solutions 1 and 2 in step b) of the method is effected at a temperature in the range of from 0° C. to 100° C., preferably in the range of from 10° C. to 95° C., particularly preferably in the range of from 15° C. to 80° C. The time for mixing the two solutions in step b) of the method is, for example, in the range of from 1 second to 6 hours, preferably in the range of from 1 minute to 2 hours. In general, mixing time in the batchwise procedure is longer than in the continuous procedure. The mixing in step b) of the method can be effected, for example, by combining an aqueous solution of a copper salt, for example a copper acetate or copper nitrate, with an aqueous solution of a mixture of a polyacrylate and oxalic acid. Alternatively, an aqueous solution of a mixture of a polyacrylate and a copper salt, for example of copper acetate or copper nitrate, can also be combined with an aqueous oxalic acid solution. Furthermore, an aqueous solution of a mixture of a polyacrylate and a copper salt, for example of copper acetate or copper nitrate, can also be combined with an aqueous solution of a mixture of a polyacrylate and oxalic acid.

In a preferred embodiment of the invention, the mixing in step b) of the method is effected by metering an aqueous solution of a mixture of a polyacrylate and oxalic acid into an aqueous solution of a mixture of polyacrylate and a copper salt, for example a copper acetate or a copper nitrate, or by metering an aqueous oxalic acid solution into an aqueous solution of a mixture of a polyacrylate and a copper salt, for example of copper acetate or copper nitrate.

During the mixing or after the mixing, the surface-modified nanoparticulate copper compounds which form an aqueous suspension result. Preferably, the mixing is effected with simultaneous stirring of the mixture. After the two solutions 1 and 2 have been completely combined, the stirring is preferably continued for a time in the range from 30 minutes to 5 hours at a temperature in the range from 0° C. to 100° C.

In a further preferred embodiment of the method according to the invention, at least one of the steps a) to d) of the method is carried out continuously. In the continuously operated procedure, step b) of the method is preferably carried out in a tubular reactor.

If required, the aqueous dispersion formed in step b) can be concentrated in step c) of the method, for example if a higher solids content is desired. The concentration can be carried out in a manner known per se, for example by distilling off the water (at atmospheric pressure or at reduced pressure), filtering or centrifuging. Furthermore, it may be necessary to separate byproducts in step c) of the method from the aqueous dispersion formed in step b), namely and these would interfere with the further use of the dispersion. Suitable byproducts are primarily salts which are dissolved in water and form in the reaction according to the invention between the solutions 1 and 2, in addition to the desired surface-modified nanoparticulate copper compound, for example sodium chloride, sodium nitrate or ammonium chloride. Such byproducts can be substantially removed from the aqueous dispersion, for example, by means of a membrane method, such as nanofiltration, ultrafiltration, microfiltration or crossflow filtration.

In the optional step d) of the method, the filter cake obtained can be dried in a manner known per se, for example by spray drying or in a drying oven at temperatures of from 40 to 100° C. (preferably from 50 to 80° C. under atmospheric pressure to constant weight).

In the method according to the invention, copper salt particles are used in an effective amount. The expression “effective amount” means an amount of the copper particles which is sufficient for controlling phytophatogenic microorganisms, in particular fungi and bacteria (especially fungi), on the crops to be protected or on the seeds and does not lead to considerable damage to the treated crops or seeds. Such an amount may vary within a wide range as influenced by numerous factors, such as, for example, the pathogen to be controlled, the plant treated in each case, the climatic conditions. The effective amount of the copper salt particles is usually based on the amount of Cu²⁺ ions. Preferably, the effective amount is in the range of from 1 to 1000 g/ha, particularly preferably from 10 to 500 g/ha, in particular from 20 to 300 g/ha and especially from 50 to 200 g/ha. In the treatment of plant propagation materials, e.g. seed, in general amounts of from 0.1 to 1000 g/100 kg of propagation material or seed, preferably from 1 to 1000 g/100 kg, particularly preferably from 1 to 100 g/100 kg, in particular from 5 to 100 g/100 kg are used.

The method according to the invention for controlling phytopathogenic microorganisms is preferably effected by treating the crops to be protected from pathogens, the plant propagation material and/or the pathogens on the crop to be protected or on the plant propagation material with an effective amount of the copper salt particles. Particularly preferably, the crop to be protected from pathogen infestation and/or the pathogens on the crop to be protected is or are treated with an effective amount of the copper salt particles. The treatment is preferably effected by spray application.

The method according to the invention and the copper salt particles according to the invention are particularly suitable as fungicides for controlling harmful fungi. They are distinguished by outstanding efficiency against a broad spectrum of phytopathogenic fungi, including soil-borne pathogens, which originate in particular from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some of them are systemically active and can be used in crop protection as foliar fungicides, as fungicides for seed dressing and as soil fungicides. In addition, they are suitable for controlling fungi which, inter alia, attack wood or the roots of plants. They can be applied both before and after the infection of the plants, plant propagation materials, e.g. seeds, by the fungi. Preferably, they are applied before the infection of plants (i.e. protectively). Plant propagation materials can be treated prophylactically together with or before sowing or together with or before transplantation.

The method according to the invention and the copper salt particles according to the invention are suitable for controlling bacteria (such as Pseudomona spec., Erwinia spec., Xanthomonas spec., Rhizobium spec., Agrobacterium spec., Rhizomonas spec., Clavibacter spec., Streptomyces spec.) in a very wide range of crops. The control of bacteria is preferably effected in the cultivation of fruit and vegetables. Examples are Pseudomona spec. on tobacco, potatoes, tomatoes and leguminous plants, and Erwinia spec. on fruit, vegetables and potatoes.

Examples of crops or cereals, for example wheat, rye, barley, triticale, oats or rice; beets, for example sugar beets or fodder beets; pomaceous fruits, stone fruits and soft fruits, for example apples, pears, plums, peaches, almonds, cherries, strawberries, raspberries, currants or gooseberries; leguminous plants, for example beans, lentils, peas, lucerne or soybeans; oil plants, for example oilseed rape, mustard, olives, sunflowers, coconut, cocoa, castor beans, oil palms, peanuts or soybeans; cucurbits, for example pumpkins, cucumbers or melons; fiber plants, for example cotton, flax, hemp or jute; citrus fruits, for example oranges, lemons, grapefruits or mandarins; vegetable plants, for example spinach, lettuce, asparagus, cabbage plants, carrots, onions, tomatoes, potatoes, pumpkins or bell peppers; laurel plants, for example avocados, cinnamon or camphor; energy and raw-material plants, for example corn, soybeans, wheat, oilseed rape, sugar cane or oil palms; corn; tobacco; nuts; coffee; tea; bananas; grapevines (grapes for eating and grapes for winemaking); hops; grass, for example lawns; rubber plants; ornamental and forest plants, for example flowers, shrubs, deciduous trees and coniferous trees, and also on the propagation material, for example seeds, and on the harvested material of these plants. Further crops are agricultural crops, e.g. potatoes, sugar beet, tobacco, wheat, rye, barley, oats, rice, corn, cotton, soybeans, oilseed rape, leguminous plants, sunflowers, coffee or sugar cane; fruit, grape vines and ornamental plants and vegetable plants, e.g. cucumbers, tomatoes, beans and pumpkins, and on the propagation material, e.g. seeds, and the harvested material of these plants.

The term plant propagation materials includes all generative parts of the plant, for example seeds and vegetative plant parts, such as seedlings and tubers (for example potatoes), which can be utilized for propagating a plant. These include seeds, roots, fruits, tubers, bulbs, rhizomes, shoots and other plant parts including seedlings and young plants which are transplanted after germination or after emergence. The young plants can be protected by partial or complete treatment, for example by immersion or watering, from harmful fungi. The treatment of plant propagation materials is used for controlling a multiplicity of pathogens in cereal crops, e.g. wheat, rye, barley or oats; rice, corn, cotton and soybeans.

The term crops also includes those plants which were modified by breeding, mutagenesis or genetic engineering, including the agricultural products on the market or undergoing development. Genetically modified plants and plants whose genetic material have been modified in a manner which does not occur under natural conditions by crossing, mutations or natural recombination (i.e. reassembly of the genetic information). As a rule, one or more genes are integrated into the genetic material of the plant to improve the properties of the plant. Such genetic modifications also comprise posttranslational modifications of proteins, oligopeptides or polypeptides, for example by means of glycolylation or binding of polymers, such as, for example, prenylated, acetylated or farnelysated radicals or PEG radicals.

The method according to the invention and the copper salt particles according to the invention are especially suitable for controlling the following plant diseases: Albugo spp. (white rust) on ornamental plants, vegetable crops (for example A. candida) and sunflowers (for example A. tragopogonis); Alternaria spp. (black spot) on vegetables, oilseed rape (for example A. brassicola or A. brassicae), sugar beet (for example A. tenuis), fruit, rice, soybeans and also on potatoes (for example A. solani or A. alternata) and tomatoes (for example A. solani or A. alternata) and Alternaria spp. (black spot) on wheat; Aphanomyces spp. on sugar beet and vegetables; Ascochyta spp. on cereals and vegetables, for example A. tritici (leaf spot) on wheat and A. hordei on barley; Bipolaris and Drechslera spp. (teleomorph: Cochliobolus spp.), for example leaf spot diseases (D.maydis and B. zeicola) on corn, for example brown spot (B. sorokiniana) on cereal and, for example, B. oryzae on rice and on lawns; Blumeria (before: Erysiphe) graminis (powdery mildew) on cereals (for example wheat or barley); Botryosphaeria spp. (black dead arm disease) on grapevines (for example B. obtusa); Botrytis cinerea (teleomorph: Botryotinia fuckeliana: gray mold) on soft fruit and pomaceous fruit (inter alia strawberries), vegetables (inter alia lettuce, carrots, celery and cabbage), oilseed rape, flowers, grapevines, forest crops and wheat (ear mold); Bremia lactucae (downy mildew) on lettuce; Ceratocystis (syn. Ophiostoma) spp. (blue stain) on deciduous trees and coniferous trees, for example C. ulmi (Dutch elm disease) on elms; Cercospora spp. (Cercospora leaf spot) on corn (for example C. zeae-maydis), rice, sugar beet (for example C. beticola), sugar cane, vegetables, coffee, soybeans (for example C. sojina or C. kikuchii) and rice; Cladosporium spp. on tomatoes (for example C. fulvum: tomato leaf mold, velvet leaf spot) and cereals, for example C. herbarum (black head mold, sooty mold) on wheat; Claviceps purpurea (ergot) on cereals; Cochliobolus (anamorph: Helminthosporium or Bipolaris) spp. (leaf blotch, spot blotch) on corn (for example C. carbonum), cereals (for example C. sativus, anamorph: B. sorokiniana: brown leaf spot, spot blotch) and rice (for example C. miyabeanus, anamorph: H. oryzae); Colletotrichum (teleomorph: Glomerella) spp. (leaf blight, anthracnose) on cotton (for example C. gossypii), corn (for example C. graminicola: stalk rot and leaf blight), soft fruit, potatoes (for example C. coccodes: wilt), beans (for example C. lindemuthianum) and soybeans (for example C. truncatum); Corticium spp., for example C. sasakii (sheath blight) on rice; Corynespora cassiicola (leaf spot) on soybeans and ornamental plants; Cycloconium spp., for example C. oleaginum on olives; Cylindrocarpon spp. (for example fruit tree canker or grapevine decline, teleomorph: Nectria or Neonectria spp.) on fruit trees, grapevines (for example C. liriodendri, teleomorph: Neonectria liriodendri, black foot disease) and many ornamental trees; Dematophora (teleomorph: Rosellinia) necatrix (white root rot/ stem rot) on soybeans; Diaporthe spp. for example D. phaseolorum (stem canker) on soybeans; Drechslera (syn. Helminthosporium, teleomorph: Pyrenophora) spp. on corn, cereals, such as barley (for example D. teres, net blotch) and on wheat (for example D. tritici-repentis: tan spot), rice and lawn; Esca disease (grapevine decline, apoplexy) on grapevines caused by Formitiporia (syn. Phellinus) punctata, F. mediterranea, Phaeomoniella chlamydospora (before Phaeoacremonium chlamydosporum), Phaeoacremonium aleophilum and/or Botryosphaeria obtusa; Elsinoe spp. on pomaceous (E. pyri) and soft fruit (E. veneta: anthracnose, cane spot) and also grapevines (E. ampelina: anthracnose, birds-eye rot); Entyloma oryzae (leaf smut) on rice; Epicoccum spp. (black head mold, sooty mold) on wheat; Erysiphe spp. (powdery mildew) on sugar beet (E. betae), vegetables (for example E. pisi), such as cucumber (for example E. cichoracearum) and cabbage plants, such as oilseed rape (for example E. cruciferarum); Eutypa lata (eutypa canker or dieback, anamorph: Cytosporina lata, syn. Libertella blepharis) on fruit trees, grapevines and many ornamental trees; Exserohilum (syn. Helminthosporium) spp. on corn (for example E. turcicum); Fusarium (teleomorph: Gibberella) spp. (wilt, root and stem rot) on various plants, such as, for example, F. graminearum or F. culmorum (root rot and head blight or ear blight) on cereals (for example wheat or barley), F. oxysporum on tomatoes, F. solani on soybeans and F. verticillioides on corn; Gaeumannomyces graminis (take-all, black root rot) on cereals (for example wheat or barley) and corn; Gibberella spp. on cereals (for example G. zeae) and rice (for example G. fujikuroi: bakanae disease); Glomerella cingulata on grapevines, pomaceous fruit and other plants and G. gossypii on cotton; grain staining complex on rice; Guignardia bidwellii (black rot) on grapevines; Gymnosporangium spp. on Rosaceae and juniper, for example G. sabinae (juniper-pear rust) on pears; Helminthosporium spp. (syn. Drechslera, teleomorph: Cochliobolus) on corn, cereals and rice; Hemileia spp., for example Hemileia vastatrix (coffee leaf rust) on coffee; Isariopsis clavispora (syn. Cladosporium vitis) on grapevines; Macrophomina phaseolina (syn. phaseoli) (root/stem rot) on soybeans and cotton; Microdochium (Syn. Fusarium) nivale (snow mold) on cereals (for example wheat or barley); Microsphaera diffusa (powdery mildew) on soybeans; Monilinia spp., for example M. laxa, M. fructicola and M. fructigena (blossom blight) on stone fruit and other Rosaceae; Mycosphaerella spp. on cereals, bananas, soft fruit and peanuts, such as, for example, M. graminicola (anamorph: Septoria tritici, septoria leaf blotch) on wheat or M. fijiensis (black sigatoka disease, black leaf streak) on bananas; Peronospora spp. (downy mildew) on cabbage (for example P. brassicae), oilseed rape (for example P. parasitica), bulbous plants (for example P. destructor), tobacco (P. tabacina) and soybeans (for example P. manshurica); Phakopsora pachyrhizi and P. meibomiae (soybean rust) on soybeans; Phialophora spp., for example on grapevines (for example P. tracheiphila and P. tetraspora) and soybeans (for example P. gregata: stem disease); Phoma lingam (root and stem rot) on oilseed rape and cabbage and P. betae (leaf spot) on sugar beet; Phomopsis spp. on sunflowers, grapevines (for example P. viticola: cane and leaf spot) and soybeans (for example stem canker and pod and stem blight: P. phaseoli, teleomorph: Diaporthe phaseolorum); Physoderma maydis (brown spot disease) on corn; Phytophthora spp. (wilt, root, leaf, stem and fruit rot) on various plants, such as bell peppers and cucumber plants (for example P. capsici), soybeans (for example P. megasperma, syn. P. sojae), potatoes and tomatoes (for example P. infestans: late blight) and deciduous trees (for example P. ramorum: sudden oak death); Plasmodiophora brassicae (club root disease) on cabbage, oilseed rape, radish and other plants; Plasmopara spp., for example P. viticola (peronospora of grapevines, downy mildew) on grapevines and P. halstedii on sunflowers; Podosphaera spp. (powdery mildew) on Rosaceae, hops, pomaceous fruit and soft fruit, for example P. leucotricha on apples; Polymyxa spp., for example on cereals, such as barley and wheat (P. graminis) and sugar beet (P. betae) and the viral diseases transmitted thereby; Pseudocercosporella herpotrichoides (eye spot disease, stem break, teleomorph: Tapesia yallundae) on cereals, for example wheat or barley; Pseudoperonospora (downy mildew) on various plants, for example P. cubensis on cucumber plants or P. humili on hops; Pseudopezicula tracheiphila (red fire disease, anamorph: Phialophora) on grapevines; Puccinia spp. (rust) on various plants, for example P. triticina (brown rust of wheat), P. striiformis (stripe rust), P. hordei (dwarf leaf rust), P. graminis (stem rust, black rust) or P. recondita (brown rust of rye) on cereals, such as, for example, wheat, barley or rye, and on asparagus (for example P. asparagi); Pyrenophora (anamorph: Drechslera) tritici-repentis (tan spot) on wheat or P. teres (net blotch) on barley; Pyricularia spp., for example P. oryzae (teleomorph: Magnaporthe grisea, rice blast) on rice and P. grisea on lawn and cereals; Pythium spp. (damping-off) on lawn, rice, corn, wheat, cotton, oilseed rape, sunflowers, sugar beet, vegetables and other plants (for example P. ultimum or P. aphanidermatum); Ramularia spp., for example R. collo-cygni (leaf spot disease/physiological leaf spots) on barley and R. beticola on sugar beet; Rhizoctonia spp. on cotton, rice, potatoes, lawn, corn, oilseed rape, potatoes, sugar beet, vegetables and on various other plants, for example R. solani (root/stem rot) on soybeans, R. solani (sheath blight) on rice or R. cerealis (sharp eye spot) on wheat or barley; Rhizopus stolonifer (soft rot) on strawberries, carrots, cabbage, grapevines and tomatoes; Rhynchosporium secalis (scald) on barley, rye and triticale; Sarocladium oryzae and S. attenuatum (sheath rot) on rice; Sclerotinia spp. (stem rot or white mold) on vegetable and agricultural crops, such as oilseed rape, sunflowers (for example Sclerotinia sclerotiorum) and soybeans (for example S. rolfsii); Septoria spp. on various plants, for example S. glycines (brown spot) on soybeans, S. tritici (septoria leaf blotch) on wheat and S. (syn. Stagonospora) nodorum (leaf and glume blotch) on cereals; Uncinula (syn. Erysiphe) necator (powdery mildew, anamorph: Oidium tuckeri) on grapevines; Setosphaeria spp. (leaf blight) on corn (for example S. turcicum, syn. Helminthosporium turcicum) and lawn; Sphacelotheca spp. (smut) on corn, (for example S. reiliana: head smut), millet and sugar cane; Sphaerotheca fuliginea (powdery mildew) on cucumber plants; Spongospora subterranea (powdery scab) on potatoes and viral diseases transmitted thereby; Stagonospora spp. on cereals, for example S. nodorum (leaf and glume blotch, teleomorph: Leptosphaeria [syn. Phaeosphaeria] nodorum) on wheat; Synchytrium endobioticum on potatoes (potato wart disease); Taphrina spp., for example T. deformans (leaf curl) on peach and T. pruni (pocket plum) on plums; Thielaviopsis spp. (black root rot) on tobacco, pomaceous fruit, vegetable crops, soybeans and cotton, for example T. basicola (syn. Chalara elegans); Tilletia spp. (common or stinking bunt) on cereals, such as, for example, T. tritici (syn. T. caries, common bunt of wheat) and T. controversa (dwarf bunt) on wheat; Typhula incarnata (gray snow mold) on barley or wheat; Urocystis spp., for example U. occulta (stripe smut) on rye; Uromyces spp. (rust) on vegetable plants, such as beans (for example U. appendiculatus, syn. U. phaseoli) and sugar beet (for example U. betae); Ustilago spp. (smut) on cereals (for example U. nuda and U. avaenae), corn (for example U. maydis: corn smut) and sugar cane; Venturia spp. (scab) on apples (for example V. inaequalis) and pears; and; Verticillium spp. (wilt of leaves and shoots) on various plants, such as fruit trees and ornamental trees, grapevines, soft fruit, vegetable and agricultural crops, such as, for example, V. dahliae on strawberries, oilseed rape, potatoes and tomatoes. The method according to the invention and the copper salt particles according to the invention are especially preferred for controlling plant diseases such as Peronosporaceae, especially the Oomycentes (downy mildew, such as, for example, Plasmopara viticola, Pseudoperonospora cubensis) and Phytophthora.

In a further preferred embodiment, the method according to the invention and the copper salt particles according to the invention are suitable for controlling bacterial diseases, especially on vegetables, fruit (in particular fruit trees), tobacco, and on the seeds of these plants. They are especially suitable for controlling the following plant diseases: Pseudomona species on tobacco, potatoes, tomatoes and legumes and in particular Erwinia species on fruit, vegetables and potatoes.

The copper salt particles can be used in the types customary for agrochemical compositions, for example solutions, emulsions, suspensions, dusts, powders, pastes and granules. Preferably, the copper salt particles are used in the form of a suspension in the method. In a further preferred embodiment, the copper salt particles are used in the form of granules in the method. Particularly preferably, they are used in the form of the suspension according to the invention.

The type of composition depends on the respective intended use; it should in any case ensure a fine and uniform distribution of the compound according to the invention. Examples of types of compositions here are suspensions (SC, OD, FS), emulsifiable concentrates (EC), emulsions (EW, EO, ES), pastes, lozenges, wettable powders or dusts (WP, SP, SS, WS, DP, DS) or granules (GR, FG, GG, MG) which may either be soluble or dispersible (wettable) in water, and gels for the treatment of plant propagation materials, such as seed (GF). In general, the types of composition (for example EC, SC, OD, FS, WG, SG, WP, SP, SS, WS, GF) are used in dilute form. Types of composition such as DP, DS, GR, FG, GG and MG are as a rule used undiluted.

The agrochemical compositions may furthermore also comprise assistants customary for crop protection agents, the choice of the assistants depending on the specific application form or the active substance. Examples of suitable assistants are solvents, solid carriers, surface-active substances (such as further solubilizers, protective colloids, wetting agents and adherents), organic and inorganic thickeners, bactericides, antifreezes, antifoams, optionally dyes and adhesives (e.g. for seed treatment).

Suitable solvents are water, organic solvents such as mineral oil fractions having a medium to high boiling point, such as kerosene and diesel oil, and furthermore coal tar oils and oils of vegetable or animal origin, aliphatic, cyclic and aromatic hydrocarbons, e.g. paraffins, tetrahydronaphthalene, alkylated naphthalenes and derivatives thereof, alkylated benzenes and derivatives thereof, alcohols, such as methanol, ethanol, propanol, butanol and cyclohexanol, glycols, ketones, such as cyclohexanone, gamma-butyrolactone, dimethyl fatty acid amides, fatty acids and fatty acid esters and strongly polar solvents, for example amines, such as N-methylpyrrolidone. In principle, solvent mixtures and mixtures of the abovementioned solvents and water can also be used.

Solid carriers are mineral earths, such as silicas, silica gels, silicates, talc, kaolin, limestone, lime, chalk, bole, loess, clay, dolomite, diatomaceous earth, calcium and magnesium sulfate, magnesium oxide, ground plastics, fertilizers, such as ammonium sulfate, ammonium phosphate, ammonium nitrate, ureas and vegetable products, such as cereal meal, bark, wood and nutshell meal, cellulose powder or other solid carriers.

Suitable surface-active substances (adjuvants, wetting agents, adherents, dispersants or emulsifiers) are the alkali metal, alkaline earth metal and ammonium salts of aromatic sulfonic acids, for example of ligninsulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany) and of fatty acids, alkylsulfonates and alkylarylsulfonates, alkyl, lauryl ether and fatty alcohol sulfates, and salts of sulfated hexa-, hepta- and octadecanols, and of fatty alcohol glycol ethers, condensates of sulfonated naphthalene and its derivatives with formaldehyde, condensates of naphthalene or of naphthalenesulfonic acids with phenol and formaldehyde, polyoxyethylene octylphenol ether, ethoxylated isooctyl-, octyl- or nonylphenol, alkylphenyl and tributylphenyl polyglycol ether, alkylaryl polyether alcohols, isotridecyl alcohol, fatty alcohol-ethylene oxide condensates, ethoxylated castor oil, polyoxyethylene or polyoxypropylene alkyl ethers, lauryl alcohol polyglycol ether acetate, sorbitol esters, ligninsulfite waste liquors and proteins, denatured proteins, polysaccharides (e.g. methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and copolymers thereof.

Examples of thickeners (i.e. compounds which impart modified flow behavior to the composition, i.e. high viscosity in the rest state and low viscosity in the agitated state) are polysaccharides and organic and inorganic layer minerals, such as xanthan gum (Kelzan®, CP Kelco, USA), Rhodopol® 23 (Rhodia, France) or Veegum® (R.T. Vanderbilt, USA) or Attaclay® (Engelhard Corp., N.J., USA). Bactericides can be added for stabilizing the composition. Examples of bactericides are those based on dichlorophen and benzyl alcohol hemiformal (Proxel® from ICI or Acticide® RS from Thor Chemie and Kathon® MK from Rohm & Haas) and isothiazolinone derivatives, such as alkylisothiazolinones and benzisothiazolinones (Acticide® MBS from Thor Chemie). Examples of suitable antifreezes are ethylene glycol, propylene glycol, urea and glycerol. Examples of antifoams are silicone emulsions (such as, for example, Silikon® SRE, Wacker, Germany, or Rhodorsil®, Rhodia, France), long-chain alcohols, fatty acids, salts of fatty acids, organofluorine compounds and mixtures thereof. Examples of adhesives are polyvinylpyrrolidone, polyvinyl acetate, polyvinyl alcohol and cellulose ethers (Tylose®, Shin-Etsu, Japan).

The agrochemical compositions comprise in general from 0.01 to 95% by weight, preferably from 0.1 to 90% by weight, of the copper salt particles. The compounds are preferably used in a purity of from 90% to 100%, preferably from 95% to 100%.

For the treatment of plant propagation materials, in particular seed, water-soluble concentrates (LS), suspensions (FS), dusts (DS), water-dispersible and water-soluble powders (WS, SS), emulsions (ES), emulsifiable concentrates (EC) and gels (GF) are usually used. These compositions can be applied to the propagation materials, in particular seed, undiluted or, preferably, in dilute form. Here, the corresponding composition may be diluted from 2 to 10 times, so that from 0.01 to 60% by weight, preferably from 0.1 to 40% by weight, of active substance are present in the compositions to be used for the dressing. The application may take place before or during sowing. The treatment of plant propagation material, in particular the treatment of seed, is known to the person skilled in the art and is effected by dusting, coating, pelletizing, immersion or impregnation of the plant propagation material, the treatment preferably being effected by pelletizing, coating and dusting or by furrow treatment, so that, for example, premature germination of the seed is prevented.

The concentrations and copper salt particles in the ready-to-use preparations can be varied within relatively large ranges. In general, they are from 0.0001 to 10%, preferably from 0.01 to 1%. The active substances may also be used successfully in the ultra low volume method (ULV), it being possible to apply compositions having more than 95% by weight of active substance or even the active substance without additions.

Oils of various types, wetting agents, adjuvants, herbicides, bactericides, other fungicides and/or pesticides can be added to the active substances or to the compositions comprising them, optionally even immediately before use (tank mix). These agents can be mixed with the compositions according to the invention in a weight ratio of from 1:100 to 100:1, preferably from 1:10 to 10:1. Particularly suitable adjuvants in this context are: organically modified polysiloxanes, e.g. Break Thru S 240®; alcohol alkoxylates, e.g. Atplus® 245, Atplus® MBA 1303, Plurafac® LF 300 and Lutensol® ON 30; EO-PO block copolymers, e.g. Pluronic® RPE 2035 and Genapol® B; alcohol ethoxylates, e.g. Lutensol® XP 80; and sodium dioctylsulfosuccinate, e.g. Leophen® RA.

In the method according to the invention and in the suspension according to the invention, further agrochemical active substances can be used in addition to the copper salt particles. The following list of active substances is intended to illustrate the possible combinations but not to limit them:

A) Strobilurins:

azoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, methyl 2-(ortho-((2,5-dimethylphenyloxy-methylene)phenyl)-3-methoxyacrylate, 2-(2-(3-(2,6-dichlorophenyl)-1-methyl-allylideneaminooxymethyl)phenyl)-2-methoxyimino-N-methylacetamide;

B) Carboxamides:

carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penflufen (N-(2-(1,3-dimethylbutyl)phenyl)-1,3-dimethyl-5-fluoro-1H-pyrazole-4-carboxamide), penthiopyrad, sedaxane, tecloftalam, thifluzamide, tiadinil, 2-amino-4-methylthiazole-5-carboxanilide, N-(3′,4′, 5′-trifluorobiphenyl-2-yl)-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3,3-trimethylbutyl)-phenyl)-1,3-dimethyl-5-fluoro-1H-pyrazole-4-carboxamide;

carboxylic acid morpholides: dimethomorph, flumorph, pyrimorph;

benzamides: flumetover, fluopicolide, fluopyram, zoxamid;

other carboxamides: carpropamid, diclocymet, mandipropamid, oxytetracycline, silthiofam, N-(6-methoxypyridin-3-yl)cyclopropanecarboxamide;

C) Azoles:

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole;

imidazoles: cyazofamid, imazalil, imazalil sulfate, perfurazoate, prochloraz, triflumizole;

benzimidazoles: benomyl, carbendazim, fuberidazole, thiabendazole;

others: ethaboxam, etridiazole, hymexazole, 2-(4-chlorophenyl)-N-[4-(3,4-dimethoxy-phenyl)isoxazole-5-yl]-2-prop-2-ynyloxyacetamide;

D) Nitrogen-Containing Heterocyclyl Compounds

pyridines: fluazinam, pyrifenox, 3-[5-(4-chlorophenyl)-2,3-dimethylisoxazolidin-3-yl]-pyridine, 3-[5-(4-methylphenyl)-2,3-dimethylisoxazolidin-3-yl]pyridine;

pyrimidines: bupirimate, cyprodinil, diflumetorim, fenarimol, ferimzone, mepanipyrim, nitrapyrin, nuarimol, pyrimethanil;

piperazines: triforine;

pyrroles: fludioxonil, fenpiclonil;

morpholines: aldimorph, dodemorph, dodemorph acetate, fenpropimorph, tridemorph;

piperidines: fenpropidin;

dicarboximides: fluorimide, iprodione, procymidone, vinclozolin;

nonaromatic 5-ring heterocycles: famoxadone, fenamidon, flutianil, octhilinone, probenazole, allyl 5-amino-2-isopropyl-3-oxo-4-ortho-tolyl-2,3-dihydropyrazole-1-thiocarboxalate;

others: acibenzolar-S-methyl, amisulbrom, anilazine, blasticidin-S, captafol, captan, quinomethionate, dazomet, debacarb, diclomezine, difenzoquat, difenzoquat-methylsulfate, fenoxanil, folpet, oxolinic acid, piperalin, proquinazid, pyroquilon, quin-oxyfen, triazoxid, tricyclazole, 2-butoxy-6-iodo-3-propylchromen-4-one, 5-chloro-1-(4,6-dimethoxypyrimidin-2-yl)-2-methyl-1H-benzoimidazole, 5-chloro-7-(4-methyl-piperidin-1-yl)-6-(2,4,6-trifluorophenyl)-[1,2,4]triazolo[1,5-a]pyrimidine, 5-ethyl-6-octyl-[1,2,4]triazolo[1,5-a]pyrimidin-7-ylamine;

E) Carbamates and Dithiocarbamates

thio- and dithiocarbamates: ferbam, mancozeb, maneb, metam, methasulphocarb, metiram, propineb, thiram, zineb, ziram;

carbamates: diethofencarb, benthiavalicarb, iprovalicarb, propamocarb, propamocarb hydrochloride, valiphenal, 4-fluorophenyl N-(1-(1-(4-cyanophenyl)ethanesulfonyl)but-2-yl)carbamate;

F) Other Fungicides

guanidines: dodine, dodine free base, guazatine, guazatine acetate, iminoctadine, iminoctadine triacetate, iminoctadine tris(albesilate);

antibiotics: kasugamycin, kasugamycin hydrochloride hydrate, polyoxines, streptomycin, validamycin A;

nitrophenyl derivatives: binapacryl, dicloran, dinobuton, dinocap, nitrothal-isopropyl, tecnazen;

organometallic compounds: fentin salts, such as, for example, fentin acetate, fentin chloride, fentin hydroxide;

sulfur-containing heterocyclyl compounds: dithianon, isoprothiolane;

organophosphorus compounds: edifenphos, fosetyl, fosetyl-aluminum, iprobenfos, phosphorous acid and its salts, pyrazophos, triclofos-methyl;

organochlorine compounds: chlorthalonil, dichlofluanid, dichlorphen, flusulfamide, hexachlorobenzene, pencycuron, pentachlorophenol and salts thereof, phthalide, quintozene, thiophanate-methyl, tolylfluanide, N-(4-chloro-2-nitrophenyl)-N-ethyl-4-methylbenzenesulfonamide;

inorganic active substances: phosphorous acid and its salts, Bordeaux mixture, copper salts, such as, for example, copper acetate, copper hydroxide, copper oxy-chloride, basic copper sulfate, sulfur;

others: biphenyl, bronopol, cyflufenamid, cymoxanil, diphenylamine, metrafenone, mildiomycin, oxine-copper, prohexadione-calcium, spiroxamine, tolylfluanid, N-(cyclo-propylmethoxyimino(6-difluoromethoxy-2,3-difluorophenyl)methyl)-2-phenyl-acetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylprop-oxy)phenyl)-N-ethyl-N-methylformamidine, N′-(5-difluoromethyl-2-methyl-4-(3-tri-methylsilanylpropoxy)phenyl)-N-ethyl-N-methylformamidine, 2-{1-[2-(5-methyl-3-trifluoromethylpyrazol-1-yl)acetyl]piperidin-4-yl}thiazole-4-carboxylic acid methyl-(1,2,3,4-tetrahydronaphthalen-1-yl)amide, 2-{1-[2-(5-methyl-3-trifluoromethylpyrazol-1-yl)acetyl]piperidin-4-yl}thiazol-4-carboxylic acid methyl-(R)-1,2,3,4-tetrahydro-naphthalen-1-ylamide, 6-tert-butyl-8-fluoro-2,3-dimethylquinolin-4-ylacetate, 6-tert-butyl-8-fluoro-2,3-dimethylquinolin-4-yl methoxy acetate, N-methyl-2-{1-[2-(5-methyl-3-trifluoromethyl-1H-pyrazol-1-yl)acetyl]piperidin-4-yl}-N-[(1R)-1 ,2,3 ,4-tetrahydro-naphthalen-1-yl]-4-thiazolecarboxamide;

G) Growth Regulators

abscisic acid, amidochlor, ancymidol, 6-benzylaminopurine, brassinolid, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfid, indol-3-acetic acid, maleic hydrazide, mefluidid, mepiquat (mepiquat chloride), metconazole, naphthaleneacetic acid, N-6-benzyladenine, paclobutrazole, prohexadione (prohexadione-calcium), prohydrojasmone, thidiazuron, triapenthenol, tributyl phosphorotrithioate, 2,3,5-triiodobenzoic acid, trinexapac-ethyl and uniconazole;

H) Herbicides

acetamides: acetochlor, alachlor, butachlor, dimethachlor, dimethenamid, flufenacet, mefenacet, metolachlor, metazachlor, napropamide, naproanilide, pethoxamide, pretilachlor, propachlor, thenylchlor;

amino acid analogs: bilanafos, glyphosate, glufosinate, sulfosate;

aryloxyphenoxypropionates: clodinafop, cyhalofop-butyl, fenoxaprop, fluazifop, haloxyfop, metamifop, propaquizafop, quizalofop, quizalofop-P-tefuryl;

bipyridyls: diquat, paraquat;

carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;

cyclohexanediones: butroxydim, clethodim, cycloxydim, profoxydim, sethoxydim, tepraloxydim, tralkoxydim;

dinitroanilines: benfluralin, ethalfluralin, oryzalin, pendimethalin, prodiamine, trifluralin;

diphenyl ethers: acifluorfen, aclonifen, bifenox, diclofop, ethoxyfen, fomesafen, lactofen, oxyfluorfen;

hydroxybenzonitriles: bromoxynil, dichlobenil, ioxynil;

imidazolinones: imazamethabenz, imazamox, imazapic, imazapyr, imazaquin, imazethapyr;

phenoxyacetic acids: clomeprop, 2,4-dichlorophenoxyacetic acid (2,4-D), 2,4-DB, dichlorprop, MCPA, MCPA-thioethyl, MCPB, mecoprop;

pyrazines: chloridazon, flufenpyr-ethyl, fluthiacet, norflurazon, pyridate;

pyridines: aminopyralid, clopyralid, diflufenican, dithiopyr, fluridone, fluroxypyr, picloram, picolinafen, thiazopyr;

sulfonylureas: amidosulfuron, azimsulfuron, bensulfuron, chlorimuron-ethyl, chlorsulfuron, cinosulfuron, cyclosulfamuron, ethoxysulfuron, flazasulfuron, fluce-tosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosul-furon, pyrazosulfuron, rimsulfuron, sulfometuron, sulfosulfuron, thifensulfuron, tria-sulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxypyrimidin-2-yOurea;

triazines: ametryn, atrazin, cyanazin, dimethametryn, ethiozin, hexazinone, meta-mitron, metribuzin, prometryn, simazin, terbuthylazin, terbutryn, triaziflam;

ureas: chlorotoluron, daimuron, diuron, fluometuron, isoproturon, linuron, methabenzthiazuron,tebuthiuron;

other inhibitors of acetolactate synthase: bispyribac-sodium, cloransulam-methyl, diclosulam, florasulam, flucarbazone, flumetsulam, metosulam, orthosulfamuron, penoxsulam, propoxycarbazone, pyribambenz-propyl, pyribenzoxime, pyriftalid, pyriminobac-methyl, pyrimisulfan, pyrithiobac, pyroxasulfon, pyroxsulam;

others: amicarbazone, aminotriazole, anilofos, beflubutamide, benazolin, bencarbazone, benfluresate, benzofenap, bentazone, benzobicyclone, bromacil, bromobutide, butafenacil, butamifos, cafenstrole, carfentrazone, cinidon-ethyl, chlorthal, cinmethylin, clomazone, cumyluron, cyprosulfamide, dicamba, difenzoquat, diflufenzopyr, Drechslera monoceras, endothal, ethofumesate, etobenzanide, fentrazamide, flumiclorac-pentyl, flumioxazin, flupoxam, fluorochloridone, flurtamon, indanofan, isoxaben, isoxaflutol, lenacil, propanil, propyzamide, quinclorac, quinmerac, mesotrione, methylarsenic acid, naptalam, oxadiargyl, oxadiazone, oxazi-clomefon, pentoxazone, pinoxaden, pyraclonil, pyraflufen-ethyl, pyrasulfotol, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxyethoxymethyl)-6-trifluoromethylpyridine-3-carbonyl]bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclo-propylpyrimidin-4-carboxylate, 6-chloro-3-(2-cyclopropyl-6-methylphenoxy)pyridazin-4-01, 4-amino-3-chloro-6-(4-chlorophenyI)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate;

I) Insecticides

organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalo-thrin, permethrin, prallethrin, pyrethrin land 11, resmethrin, silafluofen, tau-fluvalinate, tefluthrin, tetramethrin, tralomethrin, transfluthrin, profluthrin, dimefluthrin,

inhibitors of insect growth: a) chitin synthesis inhibitors: benzoylureas:

chlorfluazuron, cyramazine, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdyson antagonists: halofen-ozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;

nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;

GABA antagonists: endosulfan, ethiprol, fipronil, vaniliprol, pyrafluprol, pyriprol, 5-amino-1-(2,6-dichloro-4-methylphenyI)-4-sulfi namoyl-1H-pyrazole-3-thiocarbox-amide;

macrocyclic lactones: abamectin, emamectin, milbemectin, lepimectin, spinosad, spinetoram;

mitochondrial electron transport chain inhibitor (METI)lacaricides: fenazaquin, pyridaben, tebufenpyrad, tolfenpyrad, flufenerim;

METI II land III substances: acequinocyl, fluacyprim, hydramethylnon;

decouplers: chlorfenapyr;

inhibitors of oxidative phosphorylation: cyhexatin, diafenthiuron, fenbutatin oxide, propargite;

inhibitors of sloughing of insects: cryomazine;

inhibitors of mixed function oxidases: piperonyl butoxide;

sodium channel blockers: indoxacarb, metaflumizone;

others: benclothiaz, bifenazate, cartap, flonicamid, pyridalyl, pymetrozin, sulfur, thiocyclam, flubendiamide, chlorantraniliprol, cyazypyr (HGW86); cyenopyrafen, flupyrazofos, cyflumetofen, amidoflumet, imicyafos, bistrifluron and pyrifluquinazon.

Biopesticides are preferred as further agrochemical active substances. Biopesticides are generally known, for example from “The Manual of Biocontrol Agents (formerly The Biopesticide Manual”, 4th edition 2009, Ed. Leonard Copping, British Crop Protection Council. Suitable biopesticides are naturally occurring substances (such as substances/extracts from micoorganisms, algae, higher plants, marine crustaceans, or minerals) which are able to control plant diseases and pests (so-called biochemical pesticides), and microorganisms (such as bacteria, fungi, protozoa, viruses, bacteriophages, yeasts), which are able to control plant diseases and pests (so-called microbial pesticides). Suitable microorganisms are bacteria such as Bacillus subtilis, Bacillus pumllus, Bacillus thuringiensis, Pseudomonas spp., and Streptomyces spp. Such microorganisms are commercially available, for example from Agraquest under the brand name Braritone® (B. thuringiensis subspecies kurstakiBMP 123), Serenade® (B. subtilis QST 713), Ballad® plus (B. pumllus QST 2808). Furthermore, fungi such as Trichoderma spp., Beauveria bassiana, Coniothyrium minitans (commercially available as Contans® from Neudorff GmbH), Ulocladium oudemansll (e.g. BOTRY-ZEN® from BotriZen Ltd., New Zealand), or Microsphaeropsis ochracea are suitable. Suitable protozoa are, for example, Nosema locustae. Suitable viruses are, for example, baculoviruses or granuloviruses of Cydia pomonella. Suitable yeasts are, for example, Cryptococcus and Candida species. Minerals which may be used are, for example, kaolins, sodium silicates or diatomaceous earth. Suitable biochemical pesticides are, for example, extracts of Chenopodium ambrosiode (commercially available as Requiem® from Agraquest), extracts of the neem plant, or chitosan (e.g. ARMOUR-ZEN® from BotriZen Ltd., New Zealand). Preferred biopesticides are microorganisms, in particular bacteria, especially Bacillus subtilis, Bacillus pumllus and Bacillus thuringiensis.

The invention furthermore relates to an aqueous suspension of copper salt particles which have a particle diameter of from 1 to 200 nm and comprise a water-soluble polymer, the copper salt comprising an anion which is not hydroxide and forms a precipitate with copper ions, and the suspension comprising not more than 1.0% by weight of dissolved, inorganic salts. Preferred anions and water-soluble polymers are described above.

The copper salt particles are preferably amorphous.

The suspension according to the invention comprises at least 0.1 g of copper ions per kg of suspension. Preferably, it comprises at least 0.5 g/kg, particularly preferably 2.5 g/kg and in particular at least 3.5 g/kg. The content of copper ions can be determined by means of flame atomic absorption spectrometry. The copper ions are preferably copper(II) ions.

The suspension according to the invention advantageously has a particularly low content of dissolved, inorganic salts. In general, about 1 equivalent of soluble, inorganic salt per equivalent of copper salt is formed in the synthesis, one equivalent being defined as mole per charge. Very low concentrations of dissolved, inorganic salt can be achieved in the suspension by the above-described method for the preparation of copper salt particles, in particular with the aid of step e) of the method. The suspension according to the invention preferably comprises not more than 0.5 equivalent of dissolved, inorganic salts per equivalent of copper, preferably not more than 0.2, particularly preferably not more than 0.1 and in particular not more than 0.05. Dissolved, inorganic salts are in particular salts of chloride, nitrate or acetate.

The suspension according to the invention may comprise at least one further agrochemical active substance. Suitable further agrochemical active substances are described above.

The suspension according to the invention is obtainable, in particular is obtained, by a method comprising the steps

a) preparation of an aqueous solution comprising copper ions (solution 1) and of an aqueous solution comprising at least one anion which is not hydroxide and forms a precipitate with copper ions (solution 2), at least one of the two solutions 1 and 2 comprising at least one water-soluble polymer,

b) mixing of the solutions 1 and 2 prepared in step a) at a temperature in the range of from 0 to 100° C., the copper salt particles forming with formation of an aqueous suspension, and

e) removal of the dissolved, inorganic salts.

Steps a) and b) correspond to the above-described steps a) and b) of the method for the preparation of copper salt particles.

In step e), dissolved, inorganic salts are separated off, preferably by filtration, in particular by filtration by means of membrane methods. Suitable dissolved, inorganic salts are primarily salts which are dissolved in water and which, in the reaction between the solutions 1 and 2, form in addition to the desired surface-modified nanoparticulate copper compound, such as dissolved, inorganic salts (for example sodium chloride, sodium nitrate or ammonium chloride). Such byproducts can be substantially removed from the aqueous dispersion, for example, by means of a membrane method, such as nanofiltration, ultrafiltration, microfiltration or crossflow filtration. In a preferred embodiment, in step e), the byproduct is separated off by means of ultrafiltration (UF).

Preferably, concentration and removal of salt are effected by means of ultrafiltration in the concentration mode or optionally in the concentration and diafiltration mode, particularly preferably first in the concentration mode and then in the diafiltration mode.

In the ultrafiltration, the insoluble copper salt particles are virtually completely retained and excess, water-soluble polymer is partly or virtually completely retained by the membranes. Dissolved, inorganic salt, solvent and other lower molecular weight compounds pass through the membrane into the permeate. The depletion of the dissolved components which can pass through the membrane is dependent on the concentration factor MK=m_{feed}/mᵣₑₜₑₙₜₐₜₑ or m^{o}_{feed}/m^{o}ᵣₑₜₑₙₜₐₜₑ and on the retention of the component R=1-[cₚₑᵣₘₑₐₜₑ/Cᵣₑₜₑₙₜₐₜₑ] (m=mass, m^{o}=mass/time, c=concentration). However, since the concentration is as a rule limited, complete separation of the permeable components from the retentate is generally not possible. With a retention of 0 (unhindered passage), for example, a depletion of 90 or 95% is achieved, for example at a concentration factor of 10 or 20, respectively.

It is therefore possible, if the purity is insufficient, to separate off further dissolved, inorganic salt and further lower molecular weight compounds in a following diafiltration step (i.e. diafiltration mode). Permeate is separated off and the same amount of diafiltration medium (water) is run into the retentate, i.e. the amount of retentate is kept constant. The depletion of the diafiltration is dependent on the diafiltration coefficient MA=mpermeate/mretentate or=m^{o}permeate/m^{o}retentate and on the retention. With a retention of 0 (unhindered passage), for example, a depletion of 63, 87 or 95% is achieved at a diafiltration coefficient of 1, 2 or 3, respectively.

Different membranes (with regard to membrane material, cut-off and geometry) having a cut-off of from 2 to 500 kD or pore diameter from 3 to 200 nm can be used. The upper limit is determined by the molecular weight or the size of the water-soluble polymer or the size of copper salt particles. Polymer should be at least partly retained and copper salt particles should be virtually completely retained by the membrane. Virtually none of the salts formed in the reaction and virtually none of any low molecular weight secondary compounds present in the polymeric protective colloid may be retained by the membrane. The separation layers of the membranes may consist of organic polymers, ceramic, metal, carbon or combinations thereof and must be stable in the feed medium at the filtration temperature. For mechanical reasons, the separation layers are as a rule applied to a one-layer or multilayer porous substructure comprising the same materials as the separation layer or a plurality of different materials. Examples of possible material combinations are mentioned in the table below. The ceramic used may be, for example, α-Al₂O₃, ZrO₂, TiO₂, SiC or mixed ceramic materials. Suitable polymers are, for example, polyethylene, polypropylene, PTFE, PVDF, polysulfone, polyethersulfone, polyether ether ketone, polyamide, polyacrylonitrile or polyester.

| | |
|---|---|
| Separation layer | Substructure (coarser than separation layer) |
| Metal | Metal |
| Ceramic | Metal, ceramic or carbon |
| Polymer | Polymer, metal, ceramic or ceramic on metal |
| Carbon | Carbon, metal or ceramic |

The membranes can be used in flat, tubular, multichannel element, hollow fiber, capillary or helical geometry, for which corresponding pressure housings which permit separation between retentate (Cu-containing) and permeate (Cu-free filtrate) are available. Preferably, polymeric membranes are used in helical, tubular or hollow fiber geometry. For example, the following membranes can be used:

| | | Cut-off (kD) | |
|---|---|---|---|
| Manufacturer | Membrane | pore diameter (nm) | |
| Atech innovations | UF/TiO₂ on α-Al₂O₃ ^{1, 2} | 20 | kD |
| | UF/TiO₂ on α-Al₂O₃ ^{1, 2} | 50 | nm |
| Rhodia/Orelis | UF/ZrO₂—TiO₂ on carbon ¹ | 50; 150; 300 kD | |
| | MF/ZrO₂—TiO₂ on carbon ¹ | 0.14 | μm |
| Graver | UF/TiO₂ on steel ¹ | 100 | nm |
| Technologies | | | |
| Microdyn NADIR | UF/polyethersulfone or | 5-150 | kD |
| | polysulfone ³ | | |
| Berghoff | UF/PVDF ¹ | 30 | nm |
| | UF/polyethersulfone ¹ | 100 | kD |
| GE-Osmonics | UF/polysulfone ³ | 40 | nm |
| | UF/PVDF ³ | 10 | kD |
| | MF/PVDF ³ | 300 | nm |

| | | |
|---|---|---|
| Pall Schumacher | UF/TiO₂ or ZrO₂ on ceramic ^{1, 2} | 5.10 and 50 nm |
| | MF/α-Al₂O₃ on ceramic | 100 and 200 nm |

¹ tubular membrane;
² multichannel element;
³ flat membrane for helical, pocket, plate stack or other modules with moving membrane or stirred units between the membranes, PVDF: polyvinylidine fluoride.

Substantially depending on the diameter of the membrane pores, the hydrodynamic conditions, which influence the top layer build-up on the membrane surface on the retentate side, and mechanical stability of the membrane at the filtration temperature, the optimum transmembrane pressures between retentate and permeate are from 0.2 to 10 bar, preferably from 0.5 to 5 bar, depending on the membrane type. Higher transmembrane pressures lead as a rule to higher permeate flows. Where a plurality of modules are connected in the series, the transmembrane pressure for each module can be reduced by increasing the permeate pressure and hence adapted. In order to avoid a significant top layer build-up from the nonpermeable fractions on the membrane surface, which leads to a substantial decrease in the permeate flow, relative velocity between membrane and suspension of 0.5-25 m/s is generally produced by circulation by pumping, mechanical movement of the membrane or stirring units between the membranes. The operating temperature is dependent on the membrane stability and thermal stability of the synthesis suspension. Higher temperatures lead as a rule to higher permeate flows. The achievable permeate flows are greatly dependent on the membrane type and membrane geometry used, on the process conditions, on the feed composition (substantially the concentrations of the copper salt particles and of the water-soluble polymer). The flows are typically from 2 to 200 kg/m²/h. The process can be effected batchwise by repeated passage of the suspension through the membrane modules or continuously by a single passage through one or more feed and bleed stages connected in succession.

The invention furthermore relates to the use of the suspension according to the invention for controlling phytopathogenic microorganisms and/or undesired plant growth and/or undesired insect or mite infestation and/or for regulating the growth of plants by allowing the suspension to act on the respective pests, their habitat and/or the plants to be protected from the respective pest, the soil and/or undesired plants and/or the useful plants and/or their habitat. The use for controlling phytopathogenic fungi and/or bacteria, in particular fungi, is preferred.

The invention furthermore relates to the use of the suspension according to the invention for controlling undesired insect or mite infestation on plants and/or for controlling phytopathogenic microorganisms and/or for controlling undesired plant growth by treating seeds of useful plants with the suspension. The use for controlling phytopathogenic fungi and/or bacteria, in particular fungi, is preferred.

Advantages of the present invention are high fungicidal efficiency of the method and of the suspension with respect to phytopathogenic fungi. Owing to the high efficiency, the application of copper salts in the environment can be further reduced. The lower content of dissolved, inorganic salts leads to very good plant tolerability of the suspension. Moreover, little undesired or environmentally harmful inorganic salts remain on the crops or in the soil. The agrochemical formulations of the copper salt particles were very stable and easy to use. The method and the suspension lead to high resistance of the copper salt particles to rain. In the preparation of the suspension the method leads to a very low copper content in the wastewater.

The following examples illustrate the invention without limiting it.

### EXAMPLES

Cuprozin®: suspension concentrate SC comprising 460.6 g/l of copper hydroxide (corresponds to 300 g/l of active substance), commercially available as Cuprozin® from Spiess-Urania Chemicals GmbH.

Cuprosan®: wettable powder WP comprising 87.7% by weight of copper oxychloride (corresponds to 50% of active substance), commercially available as Cuprosan 500 from Bayer Cropscience.

Polycarboxylate A: aqueous solution of poly(sodium acrylate-co-polyethylene glycol methacrylate), solids content adjusted to 40% by weight, K value 30 (1% in water), pH 7 (10% strength solution in water).

Polycarboxylate B: aqueous solution of an acrylic acid-methyl polyglycol methacrylate copolymer, pH about 7.0, dynamic viscosity about 100 mPas (23° C., DIN/EN/ISO 3219), commercially available as Sokalan® HP 80 from BASF SE.

Polycarboxylate C: aqueous solution of polyacrylic acid sodium salt (35% by weight), pH about 7.0 (neat), molar mass about 15 000 g/mol (GPC, calibrated with polystyrene sulfonate), viscosity about 250 mPas (Brookfield, 23° C., neat), commercially available as Sokalan® PA 40 from BASF SE.

Polycarboxylate D: aqueous solution of modified polycarboxylate ether sodium salt, solids content adjusted to 40% by weight, K value 30 (1% dry matter in water, pH 7), pH 6 (10% strength solution in water), viscosity 200 mPas (Brookfield, 23° C., neat), commercially available as Sokalan® CP 42 from BASF SE.

Polycarboxylate E: aqueous solution of polyacrylic acid sodium salt (45% by weight), pH about 8-9 (20% aqueous solution, DIN 53785), dynamic viscosity 300-700 mPas (23° C., 100 s⁻¹, DIN EN ISO 3219), commercially available as Pigmentverteiler S from BASF SE.

The particle size distributions were measured by light scattering on the Zetasizer Nano S apparatus (from Malvern Instruments). The mean particle size is determined according to the volume fraction. The copper content was determined by means of flame atomic absorption spectrometry.

### Example 1-Synthesis

Solution 1 comprised 0.4 mol/1 of copper acetate monohydrate (79.8 g/l) and 20 g/l of polycarboxylate A. Solution 2 comprised 0.4 mol/l of oxalic acid (36 g/l) and 20 g/l of polycarboxylate A.

Two liters of solution 1 were heated to 60° C. and 2 l of solution 2 were metered in with stirring in the course of 30 minutes and then stirred for a further 15 minutes at 60° C. The blue suspension obtained was filtered first over a fluted filter (pore size 10-15 μm) and then over a filter capsule (pore size 0.45 μm). The suspension filtered in this way had a copper content of 2.9 g/kg and a mean particle size of 11 nm.

### Example 2-Synthesis

Solution 1 comprised 0.7 mol/l of copper acetate monohydrate (139.65 g/l), 0.35 mol/l of dimethyl carbonate (31.85 g/l) and 35 g/l of polycarboxylate A and was prepared at 75° C. Solution 2 comprised 0.7 mol/l of sodium hydroxide (28 g/l) and 35 g/l of polycarboxylate A.

2 l of solution 2 were metered with stirring in the course of 30 minutes into 2 l of solution 1, which was kept at 75° C., and stirring was then effected for a further 15 minutes at 75° C. The suspension obtained was filtered first over a fluted filter (pore size 10-15 μm) and then over a filter capsule (pore size 0.45 μm). The filtered suspension had a copper content of 6.5 g/kg and a mean particle size of 24 nm.

### Example 3-Synthesis

Solution 1 was prepared at 75° C. and comprised 0.7 mol/l of copper acetate monohydrate (139.65 g/l), 0.35 mol/l of dimethyl carbonate (31.85 g/l) and 35 g/l of polycarboxylate A. Solution 2 comprised 0.7 mol/l of sodium hydroxide (28 g/l) and 35 g/l of polycarboxylate A. 2 l of solution 2 were metered with stirring in the course of 30 minutes into 2 l of solution 1, which was kept at 75° C., and stirring was then effected for a further 15 minutes at 75° C. The suspension obtained was filtered first over a glass frit (pore size 3) and then over a filter capsule (pore size 0.45 μm). The filtered suspension had a copper content of 7.2 g/kg and a mean particle size of 10 nm.

### Example 4-Synthesis

Solution 1 was prepared at room temperature and comprised 0.2 mol/l of copper acetate monohydrate (39.93 g/l) and 50 g/l of polycarboxylate A. Solution 2 comprised 0.2 mol/l of sodium hydroxide (8 g/l), 0.1 mol/l Na2CO3 (10.6 g/l) and 50 g/l of polycarboxylate A. 2 l of solution 2 were metered with stirring at room temperature in the course of 30 minutes into 2 l of solution 1 and stirring was then continued for a further 15 minutes at room temperature. The suspension obtained was filtered first over a fluted filter (pore size 10-15 μm) and then over a filter capsule (pore size 0.45 μm). The filtered suspension had a copper content of 3.9 g/kg and a mean particle size of 24 nm.

### Example 5-Synthesis

Solution 1 was prepared at room temperature and comprised 0.2 mol/l of copper acetate monohydrate (39.8 g/l) and 50 g/l of polycarboxylate A. Solution 2 comprised 0.2 mol/l of sodium hydroxide (8 g/l), 0.1 mol/l of sodium carbonate (10.6 g/l) and 50 g/l of polycarboxylate A. p 3 l of solution 2 were metered with stirring at room temperature in the course of 1.5 hours into 31 of solution 1 and stirring was then continued for a further 15 minutes.

The green suspension obtained was filtered over a 0.45 μm filter. The filtered suspension had a copper content of 4.4 g Cu/kg and a mean particle size of 52 nm.

### Example 6: Filtration of the Copper-Containing Suspension

An ultrafiltration (UF) laboratory unit, as shown schematically in FIG. 1, was used for the filtration. The UF unit consisted substantially of a pump circulation which comprises the circulation container B1, the pump P1, the heat exchanger W1, the membrane module with integrated membrane M1 and the pressure control valve V2. The temperature T1, the flow F1 and the pressure measuring apparatus P1 are integrated before the membrane module. The pressure of the retentate after the membrane filter was measured by the pressure measuring apparatus P2 and regulated via the pressure control valve V2. The pressure of the permeate after the membrane filter M1 was measured by means of the pressure measuring apparatus P3 and regulated via the pressure control valve V3. The permeate flow after the membrane filter M1 was measured by the flow rate measuring apparatus F3. Permeate obtained can optionally be recycled to the circulation container B1 or collected in the permeate container B3 and weighed by means of a balance. In addition to the pump circulation, the UF unit comprised a feed container B2 from which starting or diafiltration medium can be metered by means of the pump P2 into the circulation container B1.

Different membranes were used for working up by means of ultrafiltration. Accordingly, the operating parameters, such as the temperature, the transmembrane flow, the pressure drop on the retentate side and the transmembrane pressure, were adapted. In general, the discharge from the synthesis was introduced into the circulation B1 and the ultrafiltration unit was put into operation with permeate recycling into the circulation container B1. During the concentration of the discharge from the synthesis, permeate was removed and discharge from the synthesis was metered from the feed container into the circulation so that the level in the circulation container remained constant. In the optional subsequent diafiltration, permeate was likewise removed and diafiltration medium (water) was fed in according to the amount removed, with regulation based on the level.

A sample from example 5 was concentrated using the following parameters: the membrane used was a flat polyethersulfone membrane having a cut-off of 30 kD (from Sartorius, Sartocon Slice Cassette with 0.1 m² membrane area) and ultrafiltration was effected at a temperature of 25° C. and a transmembrane pressure of 1 bar. In the first step, the discharge from the synthesis was concentrated by the factor 6.8 and diafiltration was then effected with a diafiltration coefficient of 4.

The content of dissolved, inorganic salt (in this case acetate) was reduced from 1.3 equivalents of acetate per equivalent of copper in the feed to 0.0018 in the retentate of the diafiltration.

TABLE 1
Concentrations before and after the filtration

| | | Cu | Na | Acetate |
|---|---|---|---|---|
| | SC | [% by wt.] | [% by wt.] | [% by wt.] |
| Feed | 3.62 | 0.44 | 0.48 | 1.1 |
| Retentate concentration | n.d. | 2.9 | n.d. | n.d. |
| Retentate diafiltration | 12.83 | 3.0 | 0.21 | 0.01 |

SC: solids content, determined by drying. n.d.: not determined.

### Example 7

A sample from example 5 was concentrated in a first step as described in Example 6 (concentration factor 9.9) and then subjected to diafiltration (diafiltration factor 3.0). In addition, further concentration was effected in a third step (concentration factor 1.33).

TABLE 2
Concentrations before and after the filtration

| | SC | Cu | Na | Acetate | | TOC |
|---|---|---|---|---|---|---|
| | [% by | [% by | [% by | [% by | Carbonate | [% by |
| | wt.] | wt.] | wt.] | wt.] | [% by wt.] | wt.] |
| Feed | 4.12 | 0.6 | 0.5 | 1.10 | 0.047 | 1.4 |
| Retentate | 20.48 | 5.0 | 0.5 | 0.81 | 0.22 | 7.2 |
| concentra- | | | | | | |
| tion | | | | | | |
| Retentate | 19.26 | 5.0 | 0.13 | 0.02 | 0.21 | 6.6 |
| diafiltration | | | | | | |
| Retentate | 25.30 | 6.6 | 0.16 | 0.01 | 0.27 | 8.7 |
| 2nd | | | | | | |
| concentra- | | | | | | |
| tion | | | | | | |

SC: solids content;
TOC: total organic carbon.

The content of dissolved inorganic salt (in this case acetate) was reduced from 0.98 equivalent of acetate (MW 59 g/mol) per equivalent of copper (MW 63.4 g/mol) in the feed to 0.087 in the retentate of the concentration or 0.0019 in the retentate of the diafiltration or 0.0009 in the retentate of the 2nd concentration.

### Example 8-Biological Action

The biological action of different copper-containing formulations against the fungus Plasmopara viticola was tested in a greenhouse on grapevines as follows. In each case three plants were used and in each case 50 ml of the copper-containing formulation mentioned in tables 3-5 were applied at said dose rate (concentration of Cu²⁺ in the spray liquor) to the plants by means of spraying. Seven days later, the plants were inoculated with Plasmopara viticola. For incubation, the plants were first kept for 12-24 h in the dark at 100% atmospheric humidity, then for four days at 20-22° C. under dry conditions and with 12 h of light per day, and then for 12-24 h again in the dark at 100% atmospheric humidity. Six days after inoculation, the 3-4 oldest leaves of the plants were rated (percent diseased leaf area, “% PLASVI”).

TABLE 3
Treatment at 75 ppm Cu²⁺ dose rate

| | Copper salt | Cu content of | % |
|---|---|---|---|
| | particles | formulation | PLASVI |
| V0 | untreated ^{a)} | — | 98.90 |
| 1 | Example 1 | 2.9 g/kg | 5.67 |
| 2 | Example 2 | 6.5 g/kg | 1.67 |

^{a)} not according to the invention

TABLE 4
Treatment at 150 ppm Cu²⁺ dose rate

| | Copper salt | Cu content of | | % |
|---|---|---|---|---|
| | particles | formulation | | PLASVI |
| V0 | untreated ^{a)} | — | | 98.90 |
| V1 | Copper hydroxide ^{a)} (Cuprozin liquid) | 460.3 | g/l | 8.33 |
| V2 | Cu oxychloride ^{a)} (Cuprosan) | 50% | WP | 16.67 |
| 1 | Example 1 | 2.9 | g/kg | 5.00 |
| 2 | Example 12 | 6.5 | g/kg | 0.33 |

^{a)} not according to the invention

TABLE 5
Treatment at 300 ppm Cu²⁺ dose rate

| | Copper salt | Cu content of | % |
|---|---|---|---|
| | particles | formulation | PLASVI |
| V0 | untreated ^{a)} | — | 98.90 |
| 1 | Example 1 | 2.9 g/kg | 2.33 |
| 2 | Example 2 | 6.5 g/kg | 0.00 |

^{a)} not according to the invention

The experiments showed that, for the same dose of Cu²⁺ ions of the surface-modified copper salt particles according to the invention, a smaller percentage by area of the leaves are damaged by Plasmopara viticola, i.e. they had a stronger fungicidal action.

### Example 9-Biological Action

In a second test series, the biological action of different copper-containing formulations was tested against the fungus Plasmopara viticola in a greenhouse on grapevines as in example 8. The leaves were rated after seven days (tables 6, 7).

TABLE 6
Treatment at 75 ppm Cu²⁺ dose rate

| | Copper salt | Cu content of | | % |
|---|---|---|---|---|
| | particles | formulation | | PLASVI |
| V0 | untreated ^{a)} | — | | 98.30 |
| V1 | Cu hydroxide (Cuprozin liquid) ^{a)} | 460.3 | g/l | 13.33 |
| 1 | Example 1 | 2.9 | g/kg | 6.67 |
| 2 | Example 3 | 7.1 | g/kg | 5.67 |
| 3 | Example 4 | 3.3 | g/kg | 3.00 |

^{a)} not according to the invention

TABLE 7
Treatment at 150 ppm Cu²⁺ dose rate

| | Copper salt | Cu content of | | % |
|---|---|---|---|---|
| | particles | formulation | | PLASVI |
| V0 | untreated ^{a)} | — | | 98.30 |
| V1 | Cu hydroxide (Cuprozin liquid) ^{a)} | 460.3 | g/l | 12.33 |
| 1 | Example 1 | 2.9 | g/kg | 3.67 |
| 2 | Example 2 | 6.5 | g/kg | 1.67 |
| 3 | Example 3 | 7.1 | g/kg | 2.33 |

^{a)} not according to the invention

The experiments showed that, for the same dose of Cu²⁺ ions of the surface-modified copper salt particles according to the invention, a smaller percentage by area of the leaves are damaged by Plasmopara viticola, i.e. they had a stronger fungicidal action.

### Examples 10-18

The syntheses were each carried out at room temperature. The reaction mixture formed on mixing solution 1 and solution 2 was in each case subsequently stirred for a further 15 minutes, at which point the blue suspension obtained was filtered (pore size 10-16 μm) to obtain a copper content of about 0.6% by weight. The average particle size was determined at the end of the synthesis described. The sample was subsequently concentrated by a factor of 5 via ultrafiltration in a pressure cell and then diafiltered with water (solvent exchange coefficient 2) to a final concentration of 30 g/l of copper in the diafiltration retentate (membrane similar to example 6).

### Example 10-Synthesis

Solution 1 comprised 0.19 mol/l of copper acetate monohydrate and 100 g/l of polycarboxylate B. Solution 2 comprised 0.27 mol/l of sodium hydroxide and 0.13 mol/l of sodium carbonate. 750 ml of solution 1 were initially charged at a temperature of 25° C. 750 ml of solution 2 were metered into solution 1 during 10-25 minutes with stirring. The filtered suspension had an average particle size of 26 nm.

Example 11-Synthesis

Solution 1 comprised 0.265 mol/l of copper acetate monohydrate and 99.7 g/l of polycarboxylate D. Solution 2 comprised 0.27 mol/l of sodium hydroxide and 0.13 mol/l of sodium carbonate. 750 ml of solution 1 were initially charged at a temperature of 25° C. 750 ml of solution 2 were metered into solution 1 during 10-25 minutes with stirring. The filtered suspension had an average particle size of 20 nm.

### Example 12- Synthesis

Solution 1 comprised 0.19 mol/l of copper acetate monohydrate, 100 g/l of polycarboxylate B and 0.487 mol/l of sodium acetate. Solution 2 comprised 0.27 mol/l of sodium hydroxide and 0.13 mol/l of sodium carbonate. 750 ml of solution 1 were initially charged at a temperature of 25° C. 750 ml of solution 2 were metered into solution 1 during 10-25 minutes with stirring. The filtered suspension had an average particle size of 25 nm.

### Example 13-Synthesis

Example 12 was repeated except that the sodium acetate in solution 1 was replaced by 0.666 mol/l of acetic acid. An average particle size of 13 nm was obtained.

### Example 14-Synthesis

Solution 1 comprised 0.346 mol/l of copper acetate monohydrate and 100 g/l of polycarboxylate B. Solution 2 comprised 0.348 mol/l of sodium hydroxide and 0.174 mol/l of sodium carbonate. 750 ml of solution 1 were initially charged at a temperature of 25° C. 750 ml of solution 2 were metered into solution 1 during 10 - 25 minutes with stirring. The filtered suspension had an average particle size of 40 nm.

### Example 15-Synthesis

Solution 1 comprised 0.265 mol/l of copper acetate monohydrate and 100 g/l of polycarboxalate A. Solution 2 comprised 0.348 mol/l of sodium hydroxide and 0.174 mol/l of sodium carbonate. Solution 3 comprised 0.348 mol/l of sodium hydroxide and 0.174 mol/l of sodium carbonate.

750 ml of solution 1 were initially charged at a temperature of 25° C. 750 ml of solution 2 were metered into solution 1 during 10-25 minutes with stirring. The reaction mixture formed was subsequently stirred for a further 15 minutes. Thereafter, 39.7 g of copper acetate monohydrate were added and stirred for 5 minutes. Thereafter, solution 3 was added during 25 minutes by metered addition. The filtered suspension had an average particle size of 65 nm.

### Example 16-Synthesis

Solution 1 comprised 0.265 mol/l of copper acetate monohydrate and 100 g/l of polycarboxalate A. Solution 2 comprised 0.348 mol/l of sodium hydroxide and 0.174 mol/l of sodium carbonate. Solution 3 comprised 0.348 mol/l of sodium hydroxide. Solution 4 comprised 0.348 mol/l of sodium hydroxide and 0.174 mol/l of sodium carbonate.

750 ml of solution 1 were initially charged at a temperature of 25° C. 750 ml of solution 2 were metered into solution 1 during 10-25 minutes with stirring. The reaction mixture formed was subsequently stirred for a further 15 minutes. Thereafter, 39.7 g of copper acetate monohydrate were added and stirred for 5 minutes. Thereafter, solution 3 was added during 25 minutes by metered addition. The reaction mixture formed was subsequently stirred for a further 15 minutes. The blue suspension obtained was filtered (pore size 10-16 μm). The suspension was subsequently initially charged again at a temperature of 25° C. 39.7 g of copper acetate monohydrate were added and stirred for 5 minutes. Next solution 4 was added during 25 minutes by metered addition and stirred for a further 15 minutes. The blue suspension obtained was filtered (pore size 10-16 μm) and the filtered suspension had an average particle size of 62 nm.

### Example 17- Synthesis

Example 10 was repeated except that the polycarboxylate B in solution 1 was replaced by polycarboxylate C. An average particle size of 27.5 nm was obtained.

### Example 18-Synthesis

Example 10 was repeated except that the polycarboxylate B in solution 1 was replaced by polycarboxylate E. An average particle size of 23.5 nm was obtained.

### Example 19-Biological Action

Activity against late blight on tomatoes caused by Phytophthora infestans, protective treatment (Phytin P1)

Leaves of potted tomato plants were sprayed to runoff point with an aqueous suspension in the below-reported active concentration of copper (1000 l/ha, i.e., 75 ppm active concentration corresponds to 75 g/ha). The next day, the leaves were inoculated with an aqueous sporangial suspension of Phytophthora infestans. The plants were then placed in a water vapor-saturated chamber at temperatures between 18 and 20° C. After 4-6 days, the late blight on the untreated but infected control plants had developed to such an extent that the infection could be determined visually in %. Efficacy was calculated according to Abbott (% efficacy=(X-Y)/X*100, where X is the diseased leaf area in the case of the VO control and Y is the diseased leaf area of the particular treatment).

TABLE 8
Efficacy (experimental series 1)

| | | Active | Diseased | |
|---|---|---|---|---|
| | Copper salt | concentration | leaf area | Efficacy |
| | particles | [ppm] | [%] | [%] |
| V0 | Untreated ^{a)} | — | 97 | — |
| 1 | Example 10 | 37.5 | 20 | 79 |
| | | 75 | 15 | 84 |

^{a)} not according to the invention

TABLE 9
Efficacy (experimental series 2)

| | | Active | Diseased | |
|---|---|---|---|---|
| | Copper salt | concentration | leaf area | Efficacy |
| | particles | [ppm] | [%] | [%] |
| V0 | Untreated ^{a)} | — | 83 | — |
| 1 | Example 17 | 37.5 | 30 | 64 |
| | | 75 | 20 | 76 |
| 2 | Example 18 | 37.5 | 47 | 44 |
| | | 75 | 18 | 78 |

^{a)} not according to the invention

1-18. (canceled)

19. A method for controlling phytopathogenic microorganisms by treating the crop to be protected, the soil or the plant reproduction material with an effective amount of amorphous copper salt particles, the amorphous copper salt particles being obtainable by a method comprising:
a) preparing a first aqueous solution comprising copper ions and a second aqueous solution comprising at least one anion which is not hydroxide and forms a precipitate with copper ions, at least one of the first aqueous solution and the second solution comprising at least one water-soluble polymer,
b) mixing the first aqueous solution and the second aqueous solution at a temperature in the range of from 0 to 100° C. to form an aqueous dispersion, and
c) optionally concentrating the aqueous dispersion and/or removing by-products from the aqueous dispersion,
where the copper salt particles have a primary particle diameter of from 1 to 200 nm.

20. The method of claim 19, where the effective amount amorphous copper salt particles is from 10 to 500 g of copper ions per hectare.

21. The method of claim 19, where the water-soluble polymer being a polycarboxylate.

22. The method of claim 19, where the water-soluble polymer is a polymer having a comb-like molecular structure having from 90 to 10% by weight of macromonomers and from 10 to 90% by weight of comonomers which carry deprotonatable groups.

23. The method of claim 19, where the anion is a carbonate, phosphate, hydrogen phosphate, oxalate, borate or tetraborate ion.

24. The method of claim 19, where the copper salt particles are present in the form of an aqueous suspension.

25. The method of claim 19, where the removal step is effected by ultrafiltration in concentration mode or optionally in diafiltration mode.

26. An aqueous suspension of amorphous copper salt particles comprising copper salt particles having a primary particle diameter of from 1 to 200 nm and a water soluble polymer, the copper salt particles being obtainable by a method comprising
a) preparing a first aqueous solution comprising copper ions and a second aqueous solution comprising at least one anion which is not hydroxide and forms a precipitate with copper ions, at least one of the first aqueous solution and the second aqueous solution comprising a water-soluble polymer,
b) mixing the first aqueous solution and the second aqueous solution at a temperature in the range of from 0 to 100° C. to form the aqueous suspension, and
c) removing the dissolved inorganic salts from the aqueous suspension, where the aqueous suspension comprises no more than 0.5 equivalent of dissolved inorganic salts per equivalent of copper.

27. The aqueous suspension of claim 26, where the aqueous suspension comprises at least 0.1 g of copper ions per kg of the aqueous suspension.

28. The aqueous suspension of claim 26, further comprising an agrochemical active substance.

29. The aqueous suspension of claim 26, where the water-soluble polymer is a polycarboxylate.

30. The aqueous suspension of claim 26, where the anion is a carbonate, phosphate, hydrogen phosphate, oxalate, borate or tetraborate ion.

31. The aqueous suspension of claim 26, where the removal step is effected by ultrafiltration in concentration mode or optionally in diafiltration mode.

32. The aqueous suspension of claim 26, where the inorganic salts are chloride, nitrate or acetate.

33. A method of controlling phytopathogenic microorganisms and/or undesired plant growth and/or undesired insect or mite infestation and/or for regulating the growth of plants by comprising allowing the aqueous suspension of claim 26 to act on the respective pests, their habitat and/or the plants to be protected from the respective pest, the soil and/or undesired plants and/or the useful plants and/or their habitat.

34. The method of claim 33, where the aqueous suspension comprises at least 0.1 g of copper ions per kg of the aqueous suspension.

35. The method of claim 33, where the aqueous suspension further comprises an agrochemical active substance.

36. A method of controlling undesired insect or mite infestation on plants and/or for controlling phytopathogenic microorganisms and/or for controlling undesired plant growth by treating seeds of useful plants with the aqueous suspension of claim 26.

37. The method of claim 36, where the aqueous suspension comprises at least 0.1 g of copper ions per kg of the aqueous suspension.

38. The method of claim 36, where the aqueous suspension further comprises an agrochemical active substance.
